(19) United States
(12) Patent Application Publication
(10) Pub. No.: US 2017/0058351 A1
(43) Pub. Date: Mar. 2, 2017

(54) Methods for Molecular Classification of BRCA-Like Breast and/or Ovarian Cancer

(71) Applicants: STICHTING HET NEDERLANDS KANKER INSTITUUT-ANTONI VAN LEEUWENHOEK ZIEKENHUIS, Amsterdam (NL); AGENDIA N.V., Amsterdam (NL)

(72) Inventors: Sabine Linn, Amsterdam (NL); Tesa Severson, Amsterdam (NL); Justine Kate Peeters, Amsterdam (NL); Iris Simon, Amsterdam (NL)

(21) Appl. No.: 15/039,959

(22) PCT Filed: Nov. 28, 2014
(86) PCT No.: PCT/NL2014/050813

§ 371 Date: May 27, 2016

Related U.S. Application Data
(60) Provisional application No. 61/910,063, filed on Nov. 28, 2013.

(51) Int. Cl. C12Q 1/68

(52) U.S. Cl. CPC C12Q 1/6886; C12Q 2600/118; C12Q 2600/158; C12Q 2600/154

(57) ABSTRACT

The invention relates to a method of assigning treatment to a breast and/or ovarian cancer patient. More specifically, the invention relates to a method for classification of breast and/or ovarian cancer as BRCA-like or sporadic-like by determining a level of expression of a set of genes and comparing said level of expression to a reference. A patient that is classified as BRCA-like is treated with a DNA-damage inducing agent.

### FIELD OF THE INVENTION

The invention relates to the field of oncology. More specifically, the invention relates to a method for typing breast and/or ovarian cancer cells. The invention provides means and methods for classification of breast and/or ovarian cancer cells.

### BACKGROUND OF THE INVENTION

Maintenance of DNA integrity depends on homologous recombination, a conservative mechanism for error-free repair of double strand breaks (DSBs). In the absence of homologous recombination, alternative error-prone mechanisms such as non-homologous end joining are invoked, leading to genomic instability (Karran, 2000. Curr Opin Genet Dev 10: 144-50; Khanna and Jackson, 2001. Nat Genet 27: 247-54; van Gent et al., 2001. Nat Rev Genet 2: 196-206). This instability is thought to predispose to familial breast and/or ovarian cancer in patients carrying germ line mutations in BRCA1 or BRCA2, genes involved in homologous recombination. Absence of homologous recombination offers a potential drug target for therapies that lead to DSBs during the DNA replication phase, when homologous recombination is the dominant DSB repair mechanism. Examples of these therapies are bifunctional alkylating agents, which cause DNA interstrand crosslinks resulting in direct DSBs in the DNA; platinum compounds, which give rise to mainly DNA intrastrand crosslinks resulting in DSBs during DNA replication; and poly(ADP-ribose)polymerase (PARP)-inhibitors (Bryant et al., 2005. Nature 434: 913-7; Fong et al., 2009. N Engl J Med 361: 123-34), which inhibit repair of single-strand DNA breaks also resulting in DSBs during replication. Recent evidence is indeed showing that BRCA1/-2-mutated breast cancers are particularly sensitive to such agents (Fong et al., 2009. N Engl J Med 361: 123-134; O'Shaughnessy et al., 2009. J Clin Oncol 27: 3; Silver et al., 2010. J Clin Oncol 28: 1145-1153; Tutt et al., 2010. Lancet 376: 235-44). This sensitivity is likely not restricted to BRCA1/-2-mutated breast cancers.

It is thought that up to 30% of sporadic (germline BRCA-wild type) breast cancers have defects in homologous recombination repair, a phenotype which is often referred to as ‘BRCAness’ (Turner et al., 2004. Nat Rev Cancer 4: 814-819). In order to identify sporadic breast cancers sensitive to agents which (directly or indirectly) induce DSBs, many studies have focused on BRCA1-mutated breast cancers, since this group of tumors is relatively homogenous, clustering within the basal-like, hormone-receptor and HER2-receptor negative (triple-negative (TN)) molecular subtype ('t Veer et al., 2002. Nature 415: 530; Sorlie et al., 2003. Proc Natl Acad Sci USA 100: 8418). Consequently, multiple trials with DSB-inducing agents have been performed in patients with TN breast cancer and indeed have shown excellent responses or improved outcome not only in mutation carriers (O'Shaughnessy et al., 2009. J Clin Oncol (Meeting Abstracts) 27:3; Silver et al., 2010. J Clin Oncol 28: 1145-1153).

BRCA2-mutated breast cancers show a similar distribution over the breast cancer subtypes as sporadic tumors (−70% estrogen-receptor (ER)- or progesterone-receptor (PR)-positive) (Lakhani et al., 2002. J Clin Oncol 20: 2310), and have not been studied extensively with a similar approach.

Adjuvant systemic treatment decisions for early breast and/or ovarian cancer are generally based on results of large randomized clinical trials conducted in the general breast cancer population, not taking into account the molecular heterogeneity of the disease (Early Breast Cancer Trialists Collaborative Group (EBCTCG), 2005. Lancet 365: 1687-717). With this approach some treatment strategies that are highly beneficial to a small percentage of the general breast and/or ovarian cancer population may have been discarded in the past, such as intensified alkylating therapy (Fisher et al., 1999. J Clin Oncol 17: 3374-88; Nieto and Shpall, 2009. Curr Opin Oncol 21: 150-7). To investigate this, we hypothesized that a small subgroup of breast and/or ovarian cancer patients, with tumors that resemble BRCA-mutated breast and/or ovarian cancer, might derive substantial benefit from intensified therapy with a DNA-damage inducing agent, such as an alkylating agent.

### SUMMARY OF THE INVENTION

The present inventors have developed a gene profile, termed ‘BRCAness’ profile that is indicative of the presence of a BRCA mutation in a breast and/or ovarian cancer cell, for example a sporadic breast cancer cell.

In one aspect, the invention provides a method of assigning treatment to a breast and/or ovarian cancer patient, the method comprising determining a level of expression for at least two genes that are selected from Table 1 in a relevant sample from the cancer patient, especially a breast and/or ovarian cancer patient or a ovarian cancer patient, whereby the sample comprises expression products from a cancer cell of the patient; comparing said determined level of expression of the at least two genes to the level of expression of the at least two genes in a template; typing said sample as being BRCA-like or not, based on the comparison of the determined levels of expression; and assigning DNA-damage inducing treatment to a breast and/or ovarian cancer patient of which the sample is classified as BRCA-like. Said relevant sample preferably is a breast cancer sample and/or an ovarian cancer sample.

In a preferred method according to the invention, the sample is typed by determining a level of RNA expression for at least two genes that are selected from Table 1 and comparing said determined RNA level of expression to the level of RNA expression of the at least two genes in a reference.

In one embodiment, said DNA-damage inducing treatment preferably comprises an alkylating agent, platinum salt and/or an inhibitor of poly(ADP-ribose) polymerase (PARP; collectively termed PARP inhibitor). Preferred DNA-damage inducing treatment comprises a nitrogen mustard alkylating agent, N,N′N′-triethylenethiophosphoramide and carboplatin.

In another embodiment, said DNA-damage inducing treatment preferably comprises a PARP inhibitor, preferably 2-[(2R)-2-Methylpyrrolidin-2-yl]-1H-benzimidazole-4-carboxamide dihydrochloride benzimidazole carboxamide (ABT-888).

An DNA-damage inducing treatment, comprising a PARP inhibitor, preferably ABT-888, preferably further comprises a tyrosine kinase inhibitor. Said tyrosine kinase inhibitor preferably is (2E)-N-[4-[[3-chloro-4-[(pyridin-2-yl)methoxy]phenyl]amino]-3-cyano-7-ethoxyquinolin-6-yl]-4-(dimethylamino)but-2-enamide (Neratinib).

In a preferred method according to the invention, a level of expression of at least five genes from Table 1 is determined, more preferred a level of expression of all 77 genes from Table 1, in a relevant sample from the breast and/or ovarian cancer patient.

The level of expression of at least two genes from Table 1 in a relevant sample from the breast and/or ovarian cancer patient is compared to template, wherein the template preferably is a measure of the average level of said at least two genes in at least 10 independent individuals. Said at least 10 independent individuals are preferably suffering from breast and/or ovarian cancer.

It is further preferred that a method according to the invention is combined with a method of determining a metastasizing potential of the sample from the patient including, for example, a 70 gene Amsterdam profile (MammaPrint®; (van't Veer et al., 2002. Nature 415: 530) and other multigene expression tests such as a 21 gene signature (Oncotype DX®; Paik et al., 2004. New Engl J Med 351: 2817) and EndoPredict (Filipits et al., 2011. Clinical Cancer Research 17: 6012). A method of determining a metastasizing potential of the sample is a 70 gene Amsterdam profile. It is further preferred that a method according to the invention is combined with a method of determining the molecular subtype of the samples, for example with BluePrint (Krijgsman et al., 2011. BCRT 133: 37-47) or other multigene tests for determining molecular subtypes such as PAM50 (Chia et al., 2012. Clin Cancer Res 18: 4465-4472).

### BRIEF DESCRIPTION OF THE DRAWINGS

FIG. 1

Overview of the strategy for generating the BRCAness signature.

FIG. 2

Supervised hierarchical clustering of gene expression in triple negative breast tumors. Top differentially expressed genes in the triple negative cohort (ANOVA FDR <0.0001) reveal two groups: one enriched for BRCA1-like status and one for sporadic-like status. Sample column: black is BRCA1-like and white indicates sporadic-like.

FIG. 3.

Survival analysis. We visualized the 10 year breast cancer specific survival (univariate) of the cohort with respect to BRCA1-like status using the Kaplan-Meier method. Multivariate survival analysis was performed using the Cox proportional hazards model.

FIG. 4

Scatter plot showing the AUC value (y-axis), indicative for to the identification of BRCA1-like patients, for groups of the top ranked genes (ANOVA). The x-axis displays the number of genes within the group. The red circles indicates the groups with the least errors in training of the model (N=2, 72, 77).

FIG. 5

Heatmap showing the standardized (median centered at zero) gene expression of BRCA1 and the Claudin genes represented on the Agilent chip. The samples are ordered by their BRCA1-like (DNA copy number) status (black) as depicted on the LHS of the heatmap.

### DETAILED DESCRIPTION OF THE INVENTION

The term BRCA, as is used herein, refers to the breast cancer susceptibility gene 1 (BRCA1) and breast cancer susceptibility gene 2 (BRCA2). BRCA1 and BRCA2 are human genes that are known as tumor suppressor genes. Mutation of these genes has been linked to hereditary breast and ovarian cancer. In normal cells, BRCA1 and BRCA2 help ensure the stability of the cell's genetic material (DNA) and help prevent uncontrolled cell growth. Mutation of these genes has been linked to the development of hereditary breast and ovarian cancer. According to estimates of lifetime risk, about 12 percent of women (120 out of 1,000) in the general population will develop breast and/or ovarian cancer sometime during their lives compared with about 60 percent of women (600 out of 1,000) who have inherited a harmful mutation in BRCA1 or BRCA2.

Activation of BRCA after DNA damage occurs via activation of ataxia telangiectasia mutated serine-protein kinase (ATM) or ataxia telangiectasia and Rad3 related protein kinase (ATR). These kinases phosphorylate BRCA1 directly or indirectly (via cell cycle checkpoint kinase 2 (CHK2). ATM and ATR also phosphorylate histones (H2AX), which then co-localize together with some proteins to form nuclear foci at DNA damage sites. The foci may further include the tumor protein p53-binding protein 1 (53BP1) and the nuclear factor with BRCT domains protein 1 (NFBD1), which take part in activation of CHK2. The so called MRN complex, consisting of double-strand break repair protein (Mre11), Rad50, and Nijmegen breakage syndrome 1 protein (Nibrin), is a part of these foci as well.

The term BRCA mutation, as is used herein, refers to a mutation in BRCA1 and/or BRCA2, preferably BRCA1, and/or in one or more other genes of which the protein product associates with BRCA1 and/or BRCA2 at DNA damage sites, including ATM, ATR, Chk2, H2AX, 53BP1, NFBD1, Mre11, Rad50, Nibrin, BRCA1-associated RING domain (BARD1), Abraxas, and MSH2. A mutation in one or more of these genes may result in a gene expression pattern that mimics a mutation in BRCA1 and/or BRCA2. The BRCAness profile, therefore, is indicative of the presence of a mutation in one or more of these genes in a breast and/or ovarian cancer cell.

The term BRCAness, or BRCA-like, refers to a sporadic breast and/or ovarian cancer sample that phenotypically resembles a mutation BRCA1 and BRCA2, preferably BRCA1. For example, the term BRCAness or BRCA-like refers to sporadic breast and/or ovarian cancers in which a BRCA1-like Comparative Genomic Hybridization (CGH) pattern is detected (Lips et al., 2011. Ann Oncol 22: 870-876; Vollebergh et al., 2011. Ann Oncol 22: 1561-1570), but in which no mutation of BRCA1 could be detected. Similarly, the term BRCAness or BRCA-like also refers to sporadic breast and/or ovarian cancers that show a correlation with the BRCAness profile, but in which no mutation of BRCA1 could be detected.

The term functionally inactivated, as used herein, refers to a genetic alteration that diminishes or abolishes the activity a BRCA-dependent DNA repair mechanism. Said alteration is an insertion, a point mutation, or, preferably, two or more point mutations, or a deletion in one of more genes of which the expression product is involved, preferably required, in the BRCA-dependent DNA repair mechanism. Said genes include BRCA1 and BRCA2.

The present invention therefore provides a method of assigning treatment to a breast and/or ovarian cancer patient, the method comprising determining a level of expression for at least two genes that are selected from Table 1 in a relevant sample from the breast and/or ovarian cancer patient, whereby the sample comprises expression products from a cancer cell of the patient; comparing said determined level of expression of the at least two genes to the level of expression of the at least two genes in a template; typing said sample as being BRCA-like or not, based on the comparison of the determined levels of expression; and assigning treatment comprising a DNA-damage agent to a breast and/or ovarian cancer patient of which the sample is classified as BRCA-like. The method for assigning treatment may assist in the selection of an optimal treatment of said patient by the treating physician.

Methods of classifying a sample from a breast and/or ovarian cancer patient according to the presence or absence of a BRCAness profile in a breast and/or ovarian cancer cell comprise determining the level of expression of genes from the gene profile, as indicated in Table 1. The methods of the invention allow classifying a breast and/or ovarian cancer sample into a “BRCAness” category; in cases where no mutation in BRCA1 and/or BRCA2 could be identified or no mutation analysis was performed. Therefore, the BRCAness profile allows the functional classification of a BRCA-like phenotype in a breast and/or ovarian cancer sample, in contrast to the genotypical classification that is provided by the analysis of genetic mutations in BRCA1 and/or BRCA2. As is indicated hereinabove, the BRCAness profile can also be used to classify a sample from a breast and/or ovarian cancer patient in which the BRCA-dependent DNA repair mechanism is functionally inactivated by alteration of one or more genes encoding other components of the BRCA-dependent DNA repair mechanism.

The term BRCAness, or BRCA-like, refers to the phenotypic characterization of a sample from breast and/or ovarian cancer patient that is or resembles a phenotype that is the result of genetic aberrations including aberrations in BRCA1 and/or BRCA2 genes. Said BRCAness or BRCA-like phenotype is preferably characterized by the BRCAness profile. It was found that breast and/or ovarian cancer patients with a BRCAness or BRCA-like phenotype have an improved response to treatment comprising a DNA-damage agent, compared to a breast and/or ovarian cancer patient without a BRCA-like phenotype.

BRCA1 is required for proper function of a homologous recombination (HR)-mediated DNA repair pathway and deficiency results in genomic instability. BRCA mutated tumors have a specific pattern of alterations, which has been used to develop a BRCA-like classifier to distinguish between BRCA-like breast and/or ovarian cancers and breast and/or ovarian cancers with or without a mutation in BRCA1 and/or BRCA2. The genes depicted in Table 1 were identified in a multistep analysis of samples from breast cancer patients. In a first step, 128 breast cancer samples were classified according to the presence of mutations in BRCA1 as well as a specific pattern of chromosomal aberrations according to a Multiplex Ligation-dependent Probe Amplification (MLPA) assay, to identify both BRCA1-like mutated breast cancers and sporadic cases (Lips et al., 2011. Breast Cancer Research 13: R107). A total of 61 breast cancer samples were identified to have a BRCA1-like CGH profile (8 of which actually presented with a BRCA1 mutation), A total of 67 breast cancer samples were scored as sporadic-like using the MLPA assay (of which 4 did contain mutations in BRCA1 (BRCA−)),

Subsequently, genes were identified of which the relative level of expression is indicative for either the sporadic-like phenotype or the BRCA1-like phenotype, as determined using the MLPA assay. The term relative is used to indicate that the level of expression was compared to the level of expression in a template, in this case pooled breast cancer samples. The expression of each of the genes depicted in Table 1 correlates with one of the two phenotypic subtypes. This correlation is represented as a fold change/ratio (BRCA-like/Sporadic-like), with a positive number indicating upregulation in BRCA-like and a negative number indicating downregulation in BRCA-like. For example, upregulation of GABBR2, PROM1 and/or ROPN1B is indicative of a BRCA-like phenotype, while downregulation of these genes is indicative of a Sporadic-like phenotype.

A sample comprising RNA expression products from a cancer cell of a breast and/or ovarian cancer patient is provided after the removal of all or part of a breast and/or ovarian cancer sample from the patient during surgery biopsy. For example, a sample comprising RNA may be obtained from a needle biopsy sample or from a tissue sample comprising breast and/or ovarian cancer cells that was previously removed by surgery. The surgical step of removing a relevant tissue sample, in this case a breast and/or ovarian cancer sample, from an individual is not part of a method according to the invention.

A sample from a breast and/or ovarian cancer patient comprising RNA expression products from a tumor of the patient can be obtained in numerous ways, as is known to a skilled person. For example, the sample can be freshly prepared from cells or a tissue sample at the moment of harvesting, or it can be prepared from samples that are stored at −70° C. until processed for sample preparation. Alternatively, tissues or biopsies can be stored under conditions that preserve the quality of the protein or RNA. Examples of these preservative conditions are fixation using e.g. formaline and paraffin embedding, RNase inhibitors such as RNAsin® (Pharmingen) or RNasecure® (Ambion), aqueous solutions such as RNAlater® (Assuragen; U.S. Pat. No. 0,620,4375), Hepes-Glutamic acid buffer mediated Organic solvent Protection Effect (HOPE; DE10021390), and RCL2 (Alphelys; WO04083369), and non-aquous solutions such as Universal Molecular Fixative (Sakura Finetek USA Inc.; U.S. Pat. No. 7,138,226).

RNA may be isolated from a breast tissue sample comprising breast and/or ovarian cancer cells by any technique known in the art, including but not limited to Trizol (Invitrogen; Carlsbad, Calif.), RNAqueous® (Applied Biosystems/Ambion, Austin, Tx), Qiazol® (Qiagen, Hilden, Germany), Agilent Total RNA Isolation Lits (Agilent; Santa Clara, Calif.), RNA-Bee® (Tel-Test. Friendswood, Tex.), and Maxwell™ 16 Total RNA Purification Kit (Promega; Madison, Wis.). A preferred RNA isolation procedure involves the use of Qiazol® (Qiagen, Hilden, Germany). RNA can be extracted from a whole sample or from a portion of a sample generated by, for example section or laser dissection.

The level of RNA expression of a signature gene according to the invention can be determined by any method known in the art. Methods to determine RNA levels of genes are known to a skilled person and include, but are not limited to, Northern blotting, quantitative Polymerase chain reaction (qPCR), also termed real time PCR (rtPCR), microarray analysis and RNA sequencing. The term qPCR refers to a method that allows amplification of relatively short (usually 100 to 1000 basepairs) of DNA sequences. In order to measure messenger RNA (mRNA), the method is extended using reverse transcriptase to convert mRNA into complementary DNA (cDNA) which is then amplified by PCR. The amount of product that is amplified can be quantified using, for example, TaqMan® (Applied Biosystems, Foster City, Calif., USA), Molecular Beacons, Scorpions® and SYBR® Green (Molecular Probes). Quantitative Nucleic acid sequence based amplification (qNASBA) can be used as an alternative for qPCR.

A preferred method for determining a level of RNA expression is microarray analysis. For microarray analysis, a hybridization mixture is prepared by extracting and labelling of RNA. The extracted RNA is preferably converted into a labelled sample comprising either complementary DNA (cDNA) or cRNA using a reverse-transcriptase enzyme and labelled nucleotides. A preferred labelling introduces fluorescently-labelled nucleotides such as, but not limited to, cyanine-3-CTP or cyanine-5-CTP. Examples of labelling methods are known in the art and include Low RNA Input Fluorescent Labelling Kit (Agilent Technologies), MessageAmp Kit (Ambion) and Microarray Labelling Kit (Stratagene).

A labelled sample may comprise two dyes that are used in a so-called two-colour array. For this, the sample is split in two or more parts, and one of the parts is labelled with a first fluorescent dye, while a second part is labelled with a second fluorescent dye. The labelled first part and the labelled second part are independently hybridized to a microarray. The duplicate hybridizations with the same samples allow compensating for dye bias.

More preferably, a sample is labelled with a first fluorescent dye, while a reference, for example a sample from a breast and/or ovarian cancer pool or a sample from a relevant cell line or mixture of cell lines, is labelled with a second fluorescent dye (known as dual channel). The labelled sample and the labelled reference are co-hybridized to a microarray. Even more preferred, a sample is labelled with a single fluorescent dye and hybridized to a microarray without a reference (known as single channel).

The labelled sample is hybridized against the probe molecules that are spotted on the array. A molecule in the labelled sample will bind to its appropriate complementary target sequence on the array. Before hybridization, the arrays are preferably incubated at high temperature with solutions of saline-sodium buffer (SSC), Sodium Dodecyl Sulfate (SDS) and bovine serum albumin (BSA) to reduce background due to nonspecific binding, as is known to a skilled person.

The arrays are preferably washed after hybridization to remove labelled sample that did not hybridize on the array, and to increase stringency of the experiment by reducing cross hybridization of the labelled sample to a partial complementary probe sequence on the array. An increased stringency will substantially reduce non-specific hybridization of the sample, while specific hybridization of the sample is not substantially reduced. Stringent conditions include, for example, washing steps for five minutes at room temperature 0.1× Sodium chloride-Sodium Citrate buffer (SSC)/0.005% Triton X-102. More stringent conditions include washing steps at elevated temperatures, such as 37 degrees Celsius, 45 degrees Celsius, or 65 degrees Celsius, either or not combined with a reduction in ionic strength of the buffer to 0.05×SSC or 0.01×SSC as is known to a skilled person.

Image acquisition and data analysis can subsequently be performed to produce an image of the surface of the hybridised array. For this, the slide can be dried and placed into a laser scanner to determine the amount of labelled sample that is bound to a target spot. Laser excitation yields an emission with characteristic spectra that is indicative of the labelled sample that is hybridized to a probe molecule. In addition, the amount of labelled sample can be quantified.

The level of expression, preferably mRNA expression levels of genes depicted in Table 1, are compared to levels of expression of the same genes in a template. A preferred template comprises an RNA sample from an individual suffering from breast and/or ovarian cancer, more preferred from multiple individuals suffering from breast and/or ovarian cancer. It is preferred that said multiple samples are pooled from more than 10 individuals, more preferred more than 20 individuals, more preferred more than 30 individuals, more preferred more than 40 individuals, most preferred more than 50 individuals. A most preferred template comprises a pooled RNA sample that is isolated from tissue comprising breast and/or ovarian cancer cells from multiple individuals suffering from breast and/or ovarian cancer. Said pooled RNA samples preferably are isolated from multiple individuals that were known to suffer from known BRCA-breast and/or ovarian cancer or that were known to suffer from Sporadic breast and/or ovarian cancer.

Typing of a sample can be performed in various ways. In one method, a coefficient is determined that is a measure of a similarity or dissimilarity of a sample with said template, preferably BRCA-breast and/or ovarian cancer and/or sporadic breast and/or ovarian cancer. A number of different coefficients can be used for determining a correlation between the RNA expression level in an RNA sample from an individual and a template. Preferred methods are parametric methods which assume a normal distribution of the data.

The levels of expression of genes from the BRCAness signature in a sample of a patient are preferably compared to the levels of expression of the same genes in a sporadic breast and/or ovarian cancer sample and in a BRCA1-breast and/or ovarian cancer sample, or in a collection of sporadic breast and/or ovarian cancer samples and in a collection of BRCA1-breast and/or ovarian cancer samples. Said comparison may result in an index score indicating a similarity of the determined expression levels in a sample of a patient with the expression levels in a sporadic breast and/or ovarian cancer sample and in a BRCA1-breast and/or ovarian cancer sample. For example, an index can be generated by determining a fold change/ratio between the median value of gene expression across all BRCA-like samples and the median value of gene expression across all sporadic-like samples. The significance of this fold change/ratio as being significant between the two respective groups can be tested primarily in an ANOVA (Analysis of variance) model. Univariate p-values can be calculated in the model and after multiple correction testing (Benjamini & Hochberg, 1995, JRSS, B, 57, 289-300) can be used as a threshold for determining significance that the gene expression shows a clear difference between the groups. Multivariate analysis may also be performed in adding covariates such as hormone expression, tumor stage/grade/size into the ANOVA model. Significant genes can be imputed into a prediction model such as Diagonal Linear Discriminant analysis (DLDA) to determine the minimal and most reliable group of gene signals that can predict the factor (BRCA-like status, response to therapy etc). Internal cross validation can be performed using the “leave-one-out” method to determine reliability and stability of these genes as being predictive in the model. An independent validation gene expression dataset is needed to further validate the gene signature.

An index can also be determined by Pearson or Cosine correlation, or by a coefficient of the linear diagonals, between the expression levels of the genes in a sample of a patient and the expression levels in a sample of a sporadic breast and/or ovarian cancer and the average expression levels in BRCA1 breast and/or ovarian cancer samples. The resultant scores/coefficients can be used to provide an index score. Said score may vary between +1, indicating a prefect similarity, and −1, indicating a reverse similarity. Preferably, an arbitrary threshold is used to type samples as sporadic-like or BRCA-like breast and/or ovarian cancer. More preferably, samples are classified as sporadic-like or BRCA-like breast and/or ovarian cancer based on the respective highest similarity measurement. A similarity score is preferably displayed or outputted to a user interface device, a computer readable storage medium, or a local or remote computer system.

The result of a comparison of the determined expression levels with the expression levels of the same genes in at least one template is preferably displayed or outputted to a user interface device, a computer readable storage medium, or a local or remote computer system. The storage medium may include, but is not limited to, a floppy disk, an optical disk, a compact disk read-only memory (CD-ROM), a compact disk rewritable (CD-RW), a memory stick, and a magneto-optical disk.

The expression data are preferably normalized. Normalization refers to a method for adjusting or correcting a systematic error in the measurements of detected label. Systemic bias results in variation by inter-array differences in overall performance, which can be due to for example inconsistencies in array fabrication, staining and scanning, and variation between labelled RNA samples, which can be due for example to variations in purity. Systemic bias can be introduced during the handling of the sample in a microarray experiment. In a preferred method according to the invention, the level of expression is preferably normalized using pre-processing methods such as quantile normalization.

To reduce systemic bias, the determined RNA levels are preferably corrected for background non-specific hybridization and normalized using, for example, Feature Extraction software (Agilent Technologies). Other methods that are or will be known to a person of ordinary skill in the art, such as a dye swap experiment (Martin-Magniette et al., Bioinformatics 21:1995-2000 (2005)) can also be applied to normalize differences introduced by dye bias. Normalization of the expression levels results in normalized expression values.

Conventional methods for normalization of array data include global analysis, which is based on the assumption that the majority of genetic markers on an array are not differentially expressed between samples [Yang et al., Nucl Acids Res 30: 15 (2002)]. Alternatively, the array may comprise specific probes that are used for normalization. These probes preferably detect RNA products from housekeeping genes such as glyceraldehyde-3-phosphate dehydrogenase and 18S rRNA levels, of which the RNA level is thought to be constant in a given cell and independent from the developmental stage or prognosis of said cell.

Said normalization preferably comprises previously mentioned global analysis “median centering”, in which the “centers” of the array data are brought to the same level under the assumption that the majority of genes are not changed between conditions (with median being more robust to outliers than the mean). Said normalization preferably comprises Lowess (LOcally WEighted Scatterplot Smoothing) local regression normalization to correct for both print-tip and intensity-dependent bias (for dual channel arrays) or “quantile normalization” (which transforms all the arrays to have a common distribution of intensities) for single channel arrays

In a preferred embodiment, genes are selected of which the RNA expression levels are largely constant between individual tissue samples comprising cancer cells from one individual, and between tissue samples comprising cancer cells from different individuals. It will be clear to a skilled artisan that the RNA levels of said set of normalization genes preferably allow normalization over the whole range of RNA levels. An example of a set of normalization genes is provided in WO 2008/039071, which is hereby incorporated by reference.

Said reference is preferably a RNA sample from a relevant cell line or mixture of cell lines. The RNA from a cell line or cell line mixture can be produced in-house or obtained from a commercial source such as, for example, Stratagene Human Reference RNA. A further preferred reference is an RNA sample isolated from a tissue of a healthy individual, preferably comprising breast cells. A preferred reference comprises RNA isolated and pooled from normal adjacent tissue from cancer patients, preferably breast and/or ovarian cancer patients. As an alternative, a static reference can be generated which enables performing single channel hybridizations for this test. A preferred static reference is calculated by measuring the median/mean background-subtracted level of expression (for example green-median/MeanSignal or red-median/MeanSignal) of a gene across 1-5 hybridization replicates of a probe sequence.

A breast and/or ovarian cancer patient is a patient that suffers, or is expected to suffer, from breast and/or ovarian cancer. The term “breast cancer” includes ductal carcinoma in situ, lobular carcinoma in situ, ductal carcinoma, inflammatory carcinoma and/or lobular carcinoma. A method according to the invention preferably further comprises assessment of clinical information, such as tumor size, tumor grade, lymph node status and family history. Clinical information may be determined in part by histopathological staging. Histopathological staging involves determining the extent of spread through the layers that form the lining of the duct or lobule, combined with determining of the number of lymph nodes that are affected by the cancer, and/or whether the cancer has spread to a distant organ. A preferred staging system is the TNM (for tumors/nodes/metastases) system, from the American Joint Committee on Cancer (AJCC). The TNM system assigns a number based on three categories. “T” denotes the size of the tumor, “N” the degree of lymphatic node involvement, and “M” the degree of metastasis. The method described here is stage independent and applies to all breast cancers.

The term ovarian cancer refers to a cancerous growth arising from the ovary. More than 90% of all ovarian cancers are classified as “epithelial” and are believed to arise from the surface (epithelium) of the ovary. Carriers of mutations in BRCA1 and BRCA2 genes account for 5%-13% of ovarian cancers. Ovarian cancer can be also be staged according to the AJCC/TNM system.

A DNA-damage inducing agent that is used in a method of the invention preferably comprises induces damage in the genomic DNA of a cell. Said genomic DNA damage includes base modifications, single strand breaks and, preferably, crosslinks, such as intrastrand and interstrand cross-links. A preferred genotoxic agent is selected from an alkylating agent such as nitrogen mustard, e.g. cyclophosphamide, mechlorethamine or mustine, uramustine and/or uracil mustard, melphalan, chlorambucil, ifosfamide; nitrosourea, including carmustine, lomustine, streptozocin; an alkyl sulfonate such as busulfan, an ethylenime such as N,N′N′-triethylenethiophosphoramide (thiotepa) and analogues thereof, a hydrazine/triazine such as dacarbazine, altretamine, mitozolomide, temozolomide, altretamine, procarbazine, dacarbazine and temozolomide; an intercalating agent such as a platinum-based compound like cisplatin, carboplatin, nedaplatin, oxaliplatin and satraplatin; anthracyclines such as doxorubicin, daunorubicin, epirubicin and idarubicin; mitomycin-C, dactinomycin, bleomycin, adriamycin, mithramycin, and poly ADP ribose polymerase (PARP)-inhibitors such as 3-aminobenzamide, AZD-2281, AG014699, ABT-888, and BMN-673. A further preferred DNA-damage inducing agent is provided by radiation, including ultraviolet radiation and gamma radiation.

A BRCA-like patient is preferably treated with a DNA damage-inducing agent. A preferred DNA damage-inducing agent comprises one or more alkylating agents, one or more platinum-based compounds and/or one or more PARP inhibitors. A further preferred DNA-damage inducing agent comprises one or more alkylating agents, one or more platinum-based compounds and one or more PARP inhibitors. A most preferred DNA-damage inducing agent comprises a nitrogen mustard alkylating agent, thiotepa and/or carboplatin. A most preferred DNA-damage inducing agent comprises cyclophosphamide, thiotepa and carboplatin.

A further preferred DNA-damage inducing agent comprises a PARP inhibitor such as 3-aminobenzamide, 4-(3-(1-(cyclopropanecarbonyl)piperazine-4-carbonyl)-4-fluorobenzyl)phthalazin-1(2H)-one (AZD-2281), 8-fluoro-2-{4-[(methylamino)methyl]phenyl}-1,3,4,5-tetrahydro-6H-pyrrolo[4,3,2-ef][2]benzazepin-6-one phosphate (1:1) (AG014699), 2-[(2R)-2-Methylpyrrolidin-2-yl]-1H-benzimidazole-4-carboxamide dihydrochloride benzimidazole carboxamide (ABT-888), (8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one (BMN-673), 8-Fluoro-2-{4-[(methylamino)methyl]phenyl}-1,3,4,5-tetrahydro-6H-azepino[5,4,3-cd]indol-6-one (AG 014699) and (S)-2-(4-(piperidin-3-yl)phenyl)-2H-indazole-7-carboxamide hydrochloride (MK-4827). A most preferred PARP inhibitor is ABT-888.

DNA-damage inducing treatment comprising a PARP inhibitor, preferably MK-4827, preferably further comprises a tyrosine kinase inhibitor. Said tyrosine kinase inhibitor preferably is a receptor tyrosine kinase inhibitor such as gefitinib, erlotinib, EKB-569, lap atinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, imatinib mesylate and (2E)-N-[4-[[3-chloro-4-[(pyridin-2-yl)methoxy]phenyl]amino]-3-cyano-7-ethoxyquinolin-6-yl]-4-(dimethylamino)but-2-enamide (Neratinib; Puma Biotechnology), N-[4-[(3-Chloro-4-fluorophenyl)amino]-7-[[(3S)-tetrahydro-3-furanyl]oxy]-6-quinazolinyl]-4-(dimethylamino)-2-butenamide (BIBW2992; Afatinib, Tomtovok, Tovok) and 4-[[1-[(3-Fluorophenyl)methyl]-1H-indazol-5-yl]amino]-5-methylpyrrolo[2,1-f][1,2,4]triazin-6-yl]carbamic acid (3S)-3-morpholinylmethyl ester hydrochloride (AC480; Bristol Myers Squibb/Ambit Biosciences).

Methods for providing a DNA-damage inducing agent to an individual in need thereof suffering from breast and/or ovarian cancer are known in the art. For example, cisplatin may be administered at 2 to 3 mg/kg every 3 to 4 weeks or at 20 mg/m2/day for 5 days every 3 to 4 weeks; at 40 mg-120 mg/m2 every 3 to 4 weeks. Cisplatin is preferably administered by injection or infusion, preferably by intravenous, intra-arterial or intraperitoneal injection or infusion.

For example, anthracyclins such as doxorubicin, daunorubicin, epirubicin and idarubicin are routinely administered at 40-75 mg/m2, every 3 weeks for treatment of breast and/or ovarian cancer.

For example, gamma radiation is administered in a dose that depends on the tumour type, whether radiation is given alone or with chemotherapy, before or after surgery, the success of surgery as is known to the skilled person. For example, radiation dose raging from 20-70 Gy is administered in a fraction schedule of 1.8-2 Gy per fraction. The typical treatment schedule is 5 days per week.

Said DNA-damage inducing agent is preferably administered at a high dosage, for example at 4000-6000 mg/m2 cyclophosphamide, 300-480 mg/m2 thiotepa and 1200-1600 mg/m2 carboplatin.

Said DNA-damage inducing agent is preferably administered after a series of conventional chemotherapeutic administrations comprising, for example, 5-fluorouracil, epirubicin and cyclophosphamide. Said conventional therapy may comprise 5-fluorouracil (250-500 mg/m2), epirubicin (60-90 mg/m2), and cyclophosphamide (250-500 mg/m2), which is administered every three weeks for two-five courses. Said DNA-damage inducing agent is preferably combined with radiotherapy and, in case of hormone receptor positive breast and/or ovarian cancer, an anti-oestrogen drug such as, for example, tamoxifen.

In a preferred method according to the invention, a level of RNA expression of at least five genes from Table 1 is determined, more preferred a level of RNA expression of at least ten genes from Table 1, more preferred a level of RNA expression of at least twenty genes from Table 1, more preferred a level of RNA expression of at least thirty genes from Table 1, more preferred a level of RNA expression of at least forty genes from Table 1, more preferred a level of RNA expression of at least fifty genes from Table 1, more preferred a level of RNA expression of all seventy-seven genes from Table 1.

In a preferred method according to the invention, a level of RNA expression of OGN (NM_033014; fold change −3.21) and PTGDS (NM_000954; fold change −3.15344) is determined, more preferred of OGN (NM_033014; fold change −3.21), PTGDS (NM_000954; fold change −3.15344), MFAP4 (NM_002404; fold change −3.07539), SLC40A1 (NM_014585; fold change −2.75694) and HDC (NM_002112; fold change −2.70381) is determined; more preferred of OGN (NM_033014; fold change −3.21), PTGDS (NM_000954; fold change −3.15344), MFAP4 (NM_002404; fold change −3.07539), SLC40A1 (NM_014585; fold change −2.75694), HDC (NM_002112; fold change −2.70381), CFD (NM_001928; fold change −2.69412), AMICA1 (NM_153206; fold change −2.67956), ITM2A (NM_004867; fold change −2.65539) and CLEC10A (NM_182906; fold change (−2.63642) is determined.

In a further preferred method according to the invention, a level of RNA expression of AMICA1 (NM_153206; p-value 4.95E-13) and HDC (NM_002112; p-value 5.1E-11) is determined, more preferred of AMICA1 (NM_153206; p-value 4.95E-13), HDC (NM_002112; p-value 5.1E-11) CLEC10A (NM_182906; p-value 1.34E-10), BASP1 (NM_006317; p-value 1.41E-10) and ITM2A (NM_004867; p-value 2.85E-10) is determined; more preferred of AMICA1 (NM_153206; p-value 4.95E-13), HDC (NM_002112; p-value 5.1E-11) CLEC10A (NM_182906; p-value 1.34E-10), BASP1 (NM_006317; p-value 1.41E-10), ITM2A (NM_004867; p-value 2.85E-10), LRMP (NM_006152; p-value 4.95E-10), CFD (NM_001928; p-value 5.06 E-10), CMFG (NM_001928; p-value 7.42E-10), ADRB2 (NM_000024; p-value 7.85E-10) and GIMAP7 (NM_153236; p-value 2.19E-9) is determined.

In a further preferred method according to the invention, a level of RNA expression of ROPN1 (NM_017578; fold change 7.2108) and VGLL1 (NM_016267; fold change 5.46003) is determined, more preferred of ROPN1 (NM_017578; fold change 7.2108), VGLL1 (NM_016267; fold change 5.46003), ELF5 (NM_198381; fold change 4.96581), TTYH1 (NM_020659; fold change 4.82047) and PROM1 (NM_001145850; fold change 5.09199) is determined, more preferred of ROPN1 (NM_017578; fold change 7.2108), VGLL1 (NM_016267; fold change 5.46003), ELF5 (NM_198381; fold change 4.96581), TTYH1 (NM_020659; fold change 4.82047), PROM1 (NM_001145850; fold change 5.09199), GABBR2 (NM_005458; fold change 4.00791), TFCP2L1 (NM_014553; fold change 3.91009), PLEKHB1 (NM_021200; fold change 3.40457), NRTN (NM_004558; fold change 3.39604), and PHGDH (NM_006623; fold change 3.21109) is determined.

In a further preferred method according to the invention, a level of RNA expression of NRTN (NM_004558; p-value 3.35E-14) and PLEKHB1 (NM_021200; p-value 3.39E-11) is determined, more preferred of NRTN (NM_004558; p-value 3.35E-14), PLEKHB1 (NM_021200; p-value 3.39E-11), TTK (NM_003318; p-value 5.26E-11), PHGDH (NM_006623; p-value 1.07E-10) and CENPA (NM_001809; p-value 1.51E-10) is determined, more preferred of NRTN (NM_004558; p-value 3.35E-14), PLEKHB1 (NM_021200; p-value 3.39E-11), TTK (NM_003318; p-value 5.26E-11), PHGDH (NM_006623; p-value 1.07E-10), CENPA (NM_001809; p-value 1.51E-10), VGLL1 (NM_016267; p-value 1.61E-10), TMEM38A (NM_024074; p-value 1.97E-10), ROPN1 (NM_017578; p-value 2.93E-10), DSC2 (NM_024422; p-value 3.79E-10) and ROPN1B (NM_001012337; p-value 5.01E-10) is determined.

In an further preferred method, a level of RNA expression of genes that are upregulated in a BRCA-like cancer, compared to a sporadic cancer (indicated as +), and a level of RNA expression of genes that are downregulated in a BRCA-like cancer, compared to a sporadic cancer (indicated as −), are determined, said genes comprising ROPN1 (NM_017578; fold change 7.2108) and OGN (NM_033014; fold change −3.21); ROPN1 (NM_017578; fold change 7.2108), VGLL1 (NM_016267; fold change 5.46003), OGN (NM_033014; fold change −3.21) and PTGDS (NM_000954; fold change −3.15344); ROPN1 (NM_017578; fold change 7.2108), VGLL1 (NM_016267; fold change 5.46003), ELF5 (NM_198381; fold change 4.96581), TTYH1 (NM_020659; fold change 4.82047), PROM1 (NM_001145850; fold change 5.09199), OGN (NM_033014; fold change −3.21), PTGDS (NM_000954; fold change −3.15344), MFAP4 (NM_002404; fold change −3.07539), SLC40A1 (NM_014585; fold change −2.75694) and HDC (NM_002112; fold change −2.70381), of ROPN1 (NM_017578; fold change 7.2108), VGLL1 (NM_016267; fold change 5.46003), ELF5 (NM_198381; fold change 4.96581), TTYH1 (NM_020659; fold change 4.82047), PROM1 (NM_001145850; fold change 5.09199), GABBR2 (NM_005458; fold change 4.00791), TFCP2L1 (NM_014553; fold change 3.91009), PLEKHB1 (NM_021200; fold change 3.40457), NRTN (NM_004558; fold change 3.39604), PHGDH (NM_006623; fold change 3.21109), OGN (NM_033014; fold change −3.21), PTGDS (NM_000954; fold change −3.15344), MFAP4 (NM_002404; fold change −3.07539), SLC40A1 (NM_014585; fold change −2.75694), HDC (NM_002112; fold change −2.70381), CFD (NM_001928; fold change −2.69412), AMICA1 (NM_153206; fold change −2.67956), ITM2A (NM_004867; fold change −2.65539) and CLEC10A (NM_182906; fold change (−2.63642).

A further preferred set of genes that are upregulated in a BRCA-like cancer, compared to a sporadic cancer (indicated as +), and set of genes that are downregulated in a BRCA-like cancer, compared to a sporadic cancer (indicated as −), comprise AMICA1 (NM_153206; p-value 4.95E-13) and NRTN (NM_004558; p-value 3.35E-14), AMICA1 (NM_153206; p-value 4.95E-13), HDC (NM_002112; p-value 5.1E-11), NRTN (NM_004558; p-value 3.35E-14) and PLEKHB1 (NM_021200; p-value 3.39E-11), AMICA1 (NM_153206; p-value 4.95E-13), HDC (NM_002112; p-value 5.1E-11) CLEC10A (NM_182906; p-value 1.34E-10), BASP1 (NM_006317; p-value 1.41E-10), ITM2A (NM_004867; p-value 2.85E-10), NRTN (NM_004558; p-value 3.35E-14), PLEKHB1 (NM_021200; p-value 3.39E-11), TTK (NM_003318; p-value 5.26E-11), PHGDH (NM_006623; p-value 1.07E-10) and CENPA (NM_001809; p-value 1.51E-10), and AMICA1 (NM_153206; p-value 4.95E-13), HDC (NM_002112; p-value 5.1E-11) CLEC10A (NM_182906; p-value 1.34E-10), BASP1 (NM_006317; p-value 1.41E-10), ITM2A (NM_004867; p-value 2.85E-10), LRMP (NM_006152; p-value 4.95E-10), CFD (NM_001928; p-value 5.06 E-10), CMFG (NM_001928; p-value 7.42E-10), ADRB2 (NM_000024; p-value 7.85E-10), GIMAP7 (NM_153236; p-value 2.19E-9), NRTN (NM_004558; p-value 3.35E-14), PLEKHB1 (NM_021200; p-value 3.39E-11), TTK (NM_003318; p-value 5.26E-11), PHGDH (NM_006623; p-value 1.07E-10), CENPA (NM_001809; p-value 1.51E-10), VGLL1 (NM_016267; p-value 1.61E-10), TMEM38A (NM_024074; p-value 1.97E-10), ROPN1 (NM_017578; p-value 2.93E-10), DSC2 (NM_024422; p-value 3.79E-10) and ROPN1B (NM_001012337; p-value 5.01E-10).

Yet a further preferred set of genes comprises AMICA1 (p-value 4.95E-13; fold change −2.80197) and NRTN (p-value 3.35E-14; fold change 3.67281); more preferred AMICA1 (p-value 4.95E-13; fold change −2.80197), HDC (p-value 5.10E-11; fold change −2.85068), NRTN (p-value 3.35E-14; fold change 3.67281) and PLEKHB1 (p-value 3.39E-11; 3.30942); more preferred AMICA1 (p-value 4.95E-13; fold change −2.80197), HDC (p-value 5.10E-11; fold change −2.85068), CLEC10A (p-value 1.34E-10; fold change −2.77256), NRTN (p-value 3.35E-14; fold change 3.67281), PLEKHB1 (p-value 3.39E-11; 3.30942) and TTK (p-value 5.26E-11; fold change 2.39315); more preferred AMICA1 (p-value 4.95E-13; fold change −2.80197), HDC (p-value 5.10E-11; fold change −2.85068), CLEC10A (p-value 1.34E-10; fold change −2.77256), LRMP (p-value 4.95E-10; fold change −2.20204), NRTN (p-value 3.35E-14; fold change 3.67281), PLEKHB1 (p-value 3.39E-11; 3.30942), TTK (p-value 5.26E-11; fold change 2.39315) and ROPN1 (p-value 2.93E-10; fold change 7.63253); more preferred AMICA1 (p-value 4.95E-13; fold change −2.80197), HDC (p-value 5.10E-11; fold change −2.85068), CLEC10A (p-value 1.34E-10; fold change −2.77256), LRMP (p-value 4.95E-10; fold change −2.20204), ADRB2 (p-value 7.85E-10; fold change −2.29795), NRTN (p-value 3.35E-14; fold change 3.67281), PLEKHB1 (p-value 3.39E-11; 3.30942), TTK (p-value 5.26E-11; fold change 2.39315), ROPN1 (p-value 2.93E-10; fold change 7.63253) and ROPN1B (p-value 5.01E-10; fold change 6.13033), more preferred AMICA1 (p-value 4.95E-13; fold change −2.80197), HDC (p-value 5.10E-11; fold change −2.85068), CLEC10A (p-value 1.34E-10; fold change −2.77256), LRMP (p-value 4.95E-10; fold change −2.20204), ADRB2 (p-value 7.85E-10; fold change −2.29795), ATP8A1 (p-value 8.93E-09; fold change −2.02829), LILRB5 (p-value 2.39E-08; fold change −2.3384), MIAT (p-value 1.89E-08; fold change −2.35646), TBC1D10C (p-value 6.20E-09; fold change −2.30803), NRTN (p-value 3.35E-14; fold change 3.67281), PLEKHB1 (p-value 3.39E-11; 3.30942), TTK (p-value 5.26E-11; fold change 2.39315), ROPN1 (p-value 2.93E-10; fold change 7.63253), ROPN1B (p-value 5.01E-10; fold change 6.13033), ELF5 (p-value 9.64E-10; fold change 5.25485), FAM64A (p-value 4.10E-09; fold change 2.42828), KRTCAP3 (p-value 5.21E-09; fold change 2.80703), PROM1 (p-value 6.77E-09; fold change 4.6813) and TPX2 (p-value 1.29E-09+ fold change 2.28201).

A preferred method according to the invention further comprises determining a metastasizing potential of the sample from the patient, and assigning treatment comprising a DNA-damage inducing agent to a breast and/or ovarian cancer patient of whom the sample is classified as BRCA-like and having a high metastasizing potential (poor prognosis). Said metastasizing potential is preferably determined by molecular expression profiling. Molecular expression profiling may be used instead of clinical assessment or, preferably, in addition to clinical assessment. Molecular expression profiling may facilitate the identification of patients who may be safely managed without adjuvant chemotherapy. A preferred molecular expression profiling is described in WO2002/103320, which is incorporated herein by reference. WO2002/103320 describes a molecular signature comprising at least 5 genes from a total of 231 genes that are used for determining a risk of recurrence of the breast and/or ovarian cancer. A further preferred molecular signature that is described in WO2002/103320 provides a molecular signature comprising a subset of 70 genes from the 231 genes, as depicted in Table 6 of WO2002/103320. Further preferred molecular signatures include a 21-gene recurrence score (Paik et al. N Engl J Med. 2004. 351:2817-2826) and Mammostrat™ (The Molecular Profiling Institute). A most preferred method for determining a metastasizing potential of breast cancer is a 70 gene profile (MammaPrint®) as described in Table 6 of WO2002/103320, which is incorporated herein by reference.

As an alternative, or in addition, a method according to the invention may be combined with other signatures, for example a signature for determining a molecular subtyping of the breast cancer, for example BluePrint Molecular Subtyping Profile, which classifies breast cancer into Basal-type, Luminal-type and ERBB2-type cancers as is described in U.S. patent application Ser. No. 13/546,755, which is incorporated herein by reference. Other preferred tests for determining molecular subtypes include PAM50 (Chia et al., 2012. Clin Cancer Res 18: 4465-4472).

### EXAMPLES

### Example 1

### Materials and Methods

Patient Samples 128 triple negative breast cancer samples (fresh frozen) with long-term follow-up were collected from two European cancer centers. BRCA1 mutation and promoter methylation was determined by next generation sequencing and methylation-specific multiplex ligation-dependent probe amplification (MS-MLPA) and BRCA1-like classification by MLPA [Lips et al., 2011. Breast Cancer Research 13: R107]. In addition we collected full genome expression data for all patients and mutation data for 21 known DNA repair genes. Differential gene expression was examined between tumors that classify as BRCA1-like with no mutation or methylation for mutations or dysregulation in another gene or genes involved in DNA repair, which may be responsible for the BRCA1-like phenotype. and sporadic-like.

### Gene Expression Preprocessing Methods:

### i) Exploratory Biological Analysis

The RNA quality was assessed by a Bioanalyzer and samples with RIN above 5 were selected for further analysis. RNA was amplified and labeled and hybridized to the Agendia customised Agilent whole genome microarrays according to the manufacturers protocol's.

Raw fluorescence intensities were quantified using Feature Extraction software (Agilent Technologies, Santa Clara, Calif., USA) according to the manufacturer's protocols. Quality of the microarray process is monitored by an internal Agendia QC model using QCs that are related to background issues, general array signal intensity, intensity of signature genes, product specific normalization genes, and array uniformity and control genes (positive and negative) (will provide reference to a paper). Only those samples that passed QC check were analysed further.

The Microarray expression dataset (N=128) was imported into R/Bioconductor software (www.bioconductor.org) where feature Signal intensities were pre-processed according to the LIMMA module (green channel only, R statistics) with background subtraction.

### ii) BRCAness Signature Development

### Gene Expression Normalization

After background subtraction of the single channel data, a value of 10 was added to all probe intensities. All probe intensities that were still smaller than 1 are assumed to be technical artifacts and set as missing values. The log 2 transformed probe intensities are normalized using quantile normalization [Bolstad et al., 2003. Bioinformatics 19: 185] from the R package limma in Bioconductor. Principal component analysis (PCA) showed a batch effect for biobank in triple negative. To adjust for these batches we applied ComBat [Johnson et al., 2006. Biostatistics 8: 118] without non-batch covariates. Genes with multiple probes were summarized by their first principal component or most variable probe, as described in the next section.

### Gene Summarization

Prior to summarization, missing values are filled in by 10 nearest neighbor imputation using the R package impute from Bioconductor. A gene is summarized by the first principal component of a correlating subset of its probes (all probes having a correlation higher than 0.5 with at least one other probe), or by its most variable probe if no such subset exists. When summarizing by first principal component, its sign is adjusted such that the largest element of the first loading is positive, and it is scaled to be as variable as the most variable probe. When summarizing by most variable probe, it is mean centered and missing values are restored.

For some genes, the probes do not show one single concordant signal, as might happen when they target splice variants or when a probe is defective. This discordance was measured by doing PCA and then subtracting the absolute value of the summation of the first principal component from the sum of absolute values of the first principal component. If this discordance measure is larger than 0.1, multiple signals might be present and we do not summarize the gene but keep its probes separate in further analysis. There were 43 genes (167 probes) that were seen as ‘discordant’ in the TN).

### Clustering and Visualization:

For clustering and visualization purpose in Partek genomics Suite, missing values were imputed with the median value for the gene across all samples. The data was shifted so each sample had a median of 0.0. Clustering was performed using both PCA and Hierarchical Clustering (Pearson Dissimilarity, average linkage)

Differential expression between classes was assessed using ANOVA models in Partek genomics Suite with the significant genes selected univariately with P<0.0001 and a fold change >2, or a fold change <−2.

### Supervised Analysis—Differentially Expressed Genes:

All data was filtered to have genes with variance >1 across all samples. Differential expression between classes was assessed using ANOVA models in Partek genomics Suite with the significant genes selected univariately to have any change in ‘BRCA1-like’ relative to ‘Sporadic-like’ with FDR (step up)<0.00001, Fold change >2 or Fold change <−2.

### Supervised Analysis—BRCAness Signature Development:

Top variable genes (variance >1 across all samples) were used for the model input. Genes were further filtered to include those also present in the validation set (N=2049). The Classification model was Linear Diagonal Discriminant Analysis (LDDA) with equal prior probabilities.

Gene features selected (from the top variable genes) using a univariate ANOVA examining the BRCA1-like/Sporadic-like status. Multiple groups off variables were tested from 1 to 100 in increments of 1. 1-level cross validation was predicted on the BRCA1-like status with the maximum number of partitions (“full leave-one-out”) with data randomly reordered.

The significant number of genes in the model was selected based on the Area under Curve (AUC).

### Results

A ‘BRCAness’ signature was developed using whole genome gene expression data. The signature has been developed on fresh frozen (FF) breast tumors that were categorized as either ‘BRCA1-like’ or ‘Sporadic’ using MLPA (Lips et al., 2011. Breast Cancer Research 13: R107). This prediction model endeavors to predict ‘BRCA-like’ tumors with a validated high sensitivity/specificity rate.

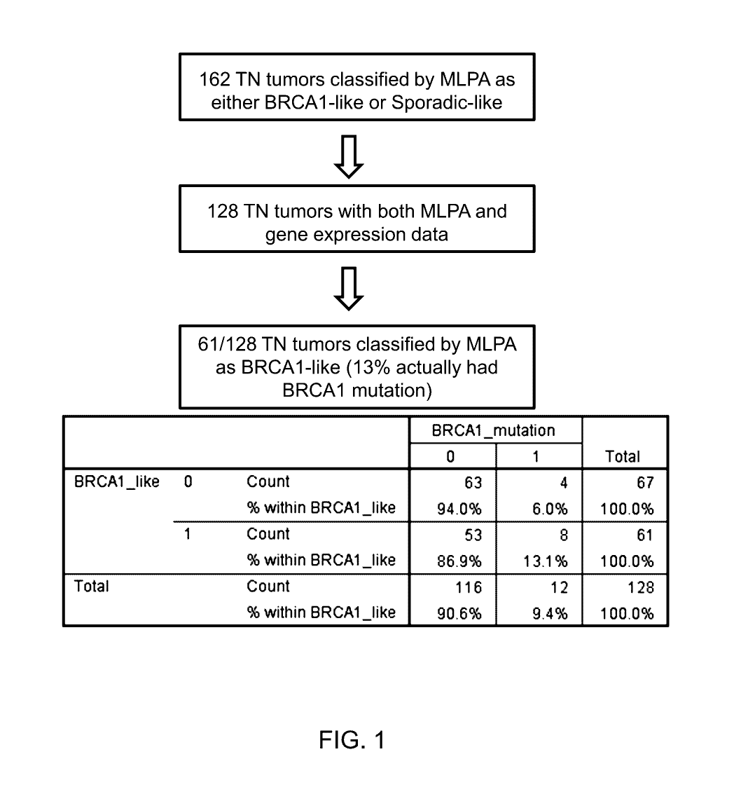

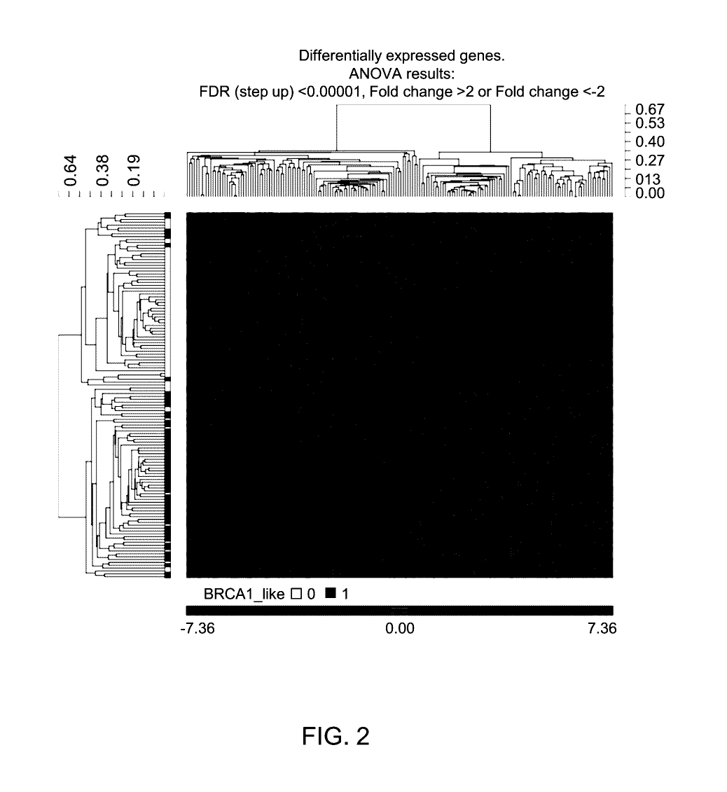

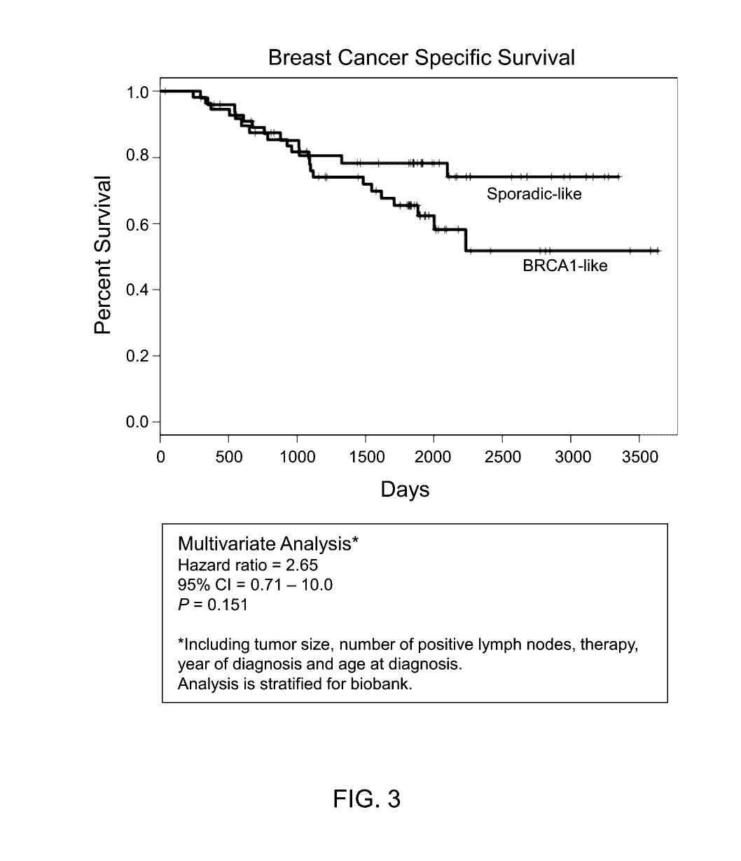

This model was built using 128 FF Triple Negative breast cancer samples (see FIG. 1). In this patient cohor, 8 (13%) of the 128 TN patients had a BRCA1 mutation. Fifty three patients were classified as BRCA1-like. Using whole genome expression analysis, we identified a set of highly significant differentially expressed genes between the BRCA1-like and sporadic-like tumors whose functions are defined as cell cycle control and DNA recombination and repair. Supervised hierarchical clustering of gene expression for this set of genes in triple negative breast tumor is shown in FIG. 2. We determined no significant differences in mutation frequency of 21 random DNA repair genes between the two classes. Breast cancer specific survival analysis (BCSS) reveals patients with a BRCA1-like tumor have a significantly worse prognosis (HR=2.25, p=0.046, CI=1.05-4.97)(see FIG. 3).

### BRCAness Signature Development

In an unsupervised analysis, 185 genes were found to be differentially expressed and were plotted using hierarchical clustering. Many of these genes were found to be involved in cell cycle control and DNA recombination and repair.

In a supervised classification model of Linear Diagonal Discriminant Analysis (LDDA), 77-gene signature was developed to identify BRCAness patients.

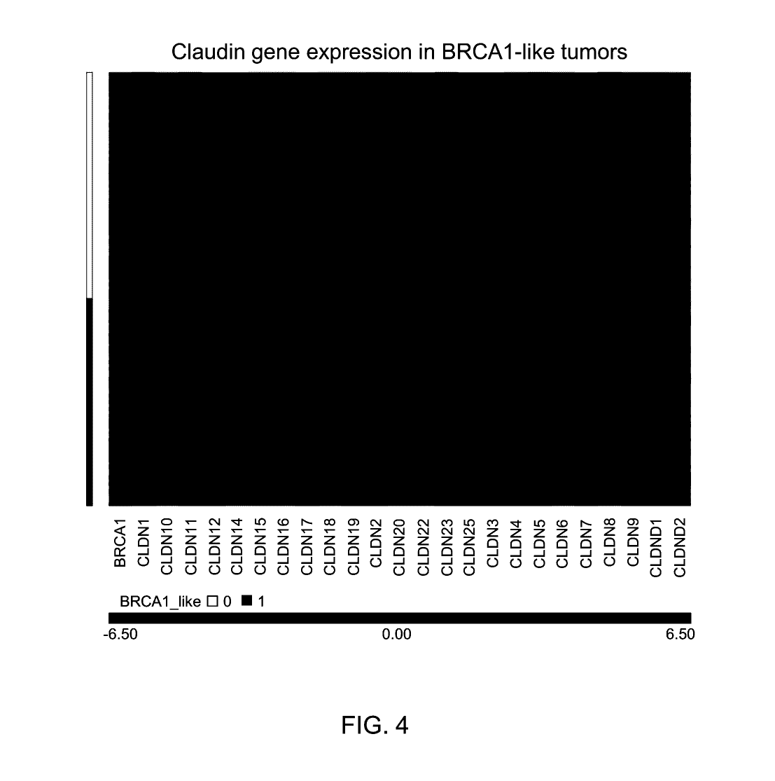

Whether the BRCAness signature is related to the Claudin-low subtype has also been explored [[Heerma van Voss et al., 2013. ASCO abstract http://meetinglibrary.asco.org/content/117999-132; Prat et al., 2010. Breast Cancer Res. 12: R68]. Heerma van Voss et al. have proposed the disregulation of the Claudin proteins in BRCA1 related tumors As is shown in FIG. 4, this is not the case for the expression of the Claudin genes in relation to the BRCA1-like status.

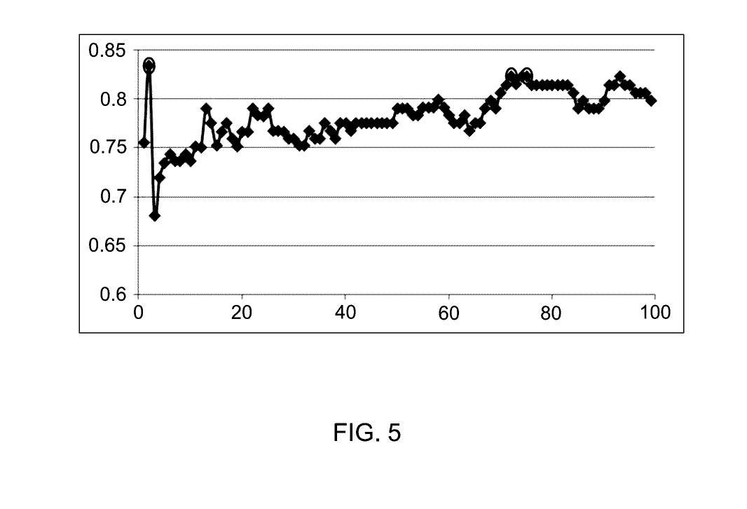

As is indicated in FIG. 5, the top 2, top 72 and top 77 genes were selected as potential signature genes.

### Example 2

A validation set comprising 53 samples was used to test the signature. This validation set had been hybridized on the Illumina microarray platform. The data for each sample was scaled to the same median as the test set.

Tables 3-6 are presented for both the training and the 53 validation samples. The top 3 significant results (2, 72 and 77 genes) are presented in Tables 3, 4 and 5, respectively Table 6 provides the results of other gene sets on the training and the 53 validation samples. For each set of genes, both results for the training dataset and the validation dataset are indicated.

Following this, a smaller number of genes were analyzed to see if there could be a ‘minimum set’ of genes that could still give the same significance in validation. The sensitivity for a lower number of genes remained the same (or even slightly higher), however the specificity dropped.

As this signature is also developed in FF a higher number of genes may be more appropriate to facilitate the conversion of the signature to FFPE. In validation of this signature, we have focused on the 77 gene panel. In the validation set, the sensitivity was 0.9200 and the specificity was 0.6071.

An update of the patient information provided in Table 5B for the validation data set resulted in a sensitivity of 0.9565, a specificity of 0.6296, a Positive Predictive Value of 0.6875, a Negative Predictive Value of 0.9444. a Matthews Correlation Coefficient of 0.6086, and an Area Under Curve of 0.7931 for the 77 gene signature.

### Conclusion

Our data show that patients with BRCA1-like tumors have a significantly worse prognosis. Although not all of these tumors are BRCA1 mutant, they do possess differentially expressed genes that are involved in cell cycle control and DNA recombination and repair and therefore may be more susceptible to specific treatments such as PARP inhibitors. A BRCAness gene signature has been developed that is able to effectively identify a group of patients that are BRCA1-like and may better respond to DNA-damage inducing agents comprising one or more alkylating agents, one or more platinum-based compounds and/or one or more PARP inhibitors.

### Example 3

### Methods

115 HER2 negative patients (HER2−) were considered in this analysis. The BRCAness classification was computed using the 77 gene panel BRCAness gene signature. Patients were treated with oral PARP inhibitor veliparib (ABT-888) in combination with carboplatin and chemotherapy (V/C) (71 patients), or with chemotherapy alone (44 patients).

The association between BRCAness classification and response in the V/C and control arms alone (Fisher Exact test), and relative performance between arms (biomarker×treatment interaction, likelihood ratio test) was determined using a logistic model. The BRCAness signature was assessed in the context of a subset of patients that were negative for progesterone receptor, estrogen receptor and HER2 (triple negative; TN). Statistical calculations are descriptive (e.g. p-values are measures of distance with no inferential content).

### Results

Of the 115 patients assessed, 56 were classified as BRCA-like using the 77 gene panel BRCAness gene signature. 16% of BRCA-like patients were progesterone receptor and estrogen receptor positive (hormone receptor positive; HR+) and HER2−.

The distribution of pathological complete response (pCR) rates among BRCAness signature dichotomized groups stratified by hormone receptor status is indicated in Table 7.

The BRCAness signature classification associated with patient response in the V/C arm (OR=6.8, p=0.0005) but not in the control arm (OR=0.75, p=1). There is a significant biomarker×treatment interaction in the V/C arm relative to control arm=9.3, p=0.018), which remains significant upon adjusting for HR status (p=0.016).

When the BRCA1-like patients were added to the graduating TN subset, the OR associated with V/C is 4.9, which is comparable to that of the TN signature (OR: 4.4), while increasing the prevalence of biomarker-positive patients by ˜8%. Evaluation of the BRCAness signature in the context of the graduating signature is pending.

Conclusion: Although the sample size was small, the analysis suggests the BRCAness signature shows promise for predicting response to veliparib/carboplatin combination therapy, relative to control. This signature will contribute to the selection criteria of PARP inhibitor trials.

TABLE 1

| | | | | | Fold- | Seq |
|---|---|---|---|---|---|---|
| Gene | mRNA | | | P- | Change | ID |
| symbol | reference | Systematic Name | Sequence | value | (BRCAness | NO |
| ABCA6 | NM_080284 | Homo sapiens ATP-binding | ATTAGTAAAGTCACCCAAAGAGTCAGGCAC | 1.07E−08 | −2.17231 | 1 |
| | | cassette, sub-family A | TGGGTATTGTGGAAATAAAACTATATAAAC | | | |
| | | (ABC1), member 6 (ABCA6), | | | | |
| | | mRNA [NM_080284] | | | | |
| ACTR3B | NM_020445 | Homo sapiens ARP3 actin- | ATAGAAGATGATGGTTTGTTGTCGGTGAGT | 1.59E−09 | 2.55945 | 2 |
| | | related protein 3 homolog | GTTGGATGAAATACTTCCTTGCACCATTGT | | | |
| | | B (yeast) (ACTR3B), trans- | | | | |
| | | cript variant 1, mRNA | | | | |
| | | [NM_020445] | | | | |
| ADRB2 | NM_000024 | Homo sapiens adrenergic, | CTCTTATTTGCTCACACGGGGTATTTTAGG | 7.85E−10 | −2.29795 | 3 |
| | | beta-2-, receptor, surface | CAGGGATTTGAGGAGCAGCTTCAGTTGTTT | | | |
| | | (ADRB2), mRNA [NM_000024] | | | | |
| AMICA1 | NM_153206 | Homo sapiens adhesion | CTCCTGTGGGCAGGGTTCTTAGTGGATGAG | 4.95E−13 | −2.80197 | 4 |
| | | molecule, interacts with | TTACTGGGAAGAATCAGAGATAAAAACCAA | | | |
| | | CXADR antigen 1 (AMICA1), | | | | |
| | | transcript variant 2, | | | | |
| | | mRNA [NM_153206] | | | | |
| ATP8A1 | NM_006095 | Homo sapiens ATPase, | CTATGCAGTGTTATGTGTCATTGGCCTTTT | 8.93E−09 | −2.02829 | 5 |
| | | aminophospholipid | GTGAATGTGCATGTTTTAAACTGCAAATTT | | | |
| | | transporter (APLT), | | | | |
| | | class I, type 8A, mem- | | | | |
| | | ber 1 (ATP8A1), trans- | | | | |
| | | cript variant 1, | | | | |
| | | mRNA [NM_006095] | | | | |
| AURKB | NM_004217 | Homo sapiens aurora | AATAGCAGTGGGACACCCGACATCTTAACG | 3.71E−08 | 2.192 | 6 |
| | | kinase B (AURKB), | CGGCACTTCACAATTGATGACTTTGAGATT | | | |
| | | mRNA [NM_004217] | | | | |
| B3GNT5 | NM_032047 | Homo sapiens UDP- | AAATGTCAACAAAGGGAAAATAAACTATCA | 1.97E−08 | 1.99447 | 7 |
| | | GlcNAc:betaGal beta- | GCTTGGATGGTCACTTGAATAGAAGATGGT | | | |
| | | 1,3-N-acetylglucos- | | | | |
| | | aminyltransferase 5 | | | | |
| | | (B3GNT5), mRNA | | | | |
| | | [NM_032047] | | | | |
| BASP1 | NM_006317 | Homo sapiens brain | TCAATGCCAATCCTCCATTCTTCCTCTCCA | 1.41E−10 | −2.05825 | 8 |
| | | abundant, membrane | GATATTTTTGGGAGTGACAAACATTCTCTC | | | |
| | | attached signal | | | | |
| | | protein 1 (BASP1), | | | | |
| | | mRNA [NM_006317] | | | | |
| C10orf35 | NM_145306 | Homo sapiens chromo | GGAGCAGGACTTGGGCTTAGGGCAGGTGGA | 9.70E−10 | 2.00989 | 9 |
| | | some 10 open reading | AAAAATTCCAGACTTTTTTAGCACTGTTTT | | | |
| | | frame 35 (C10orf35), | | | | |
| | | mRNA [NM_145306] | | | | |
| CCNA2 | NM_001237 | Homo sapiens cyclin | AAGTTTGATAGATGCTGACCCATACCTCAA | 1.36E−08 | 2.04841 | 10 |
| | | A2 (CCNA2), mRNA | GTATTTGCCATCAGTTATTGCTGGAGCTGC | | | |
| | | [NM_001237] | | | | |
| CDC20 | NM_001255 | Homo sapiens cell | GGTAATGATAACTTGGTCAATGTGTGGCCT | 1.77E−08 | 2.33461 | 11 |
| | | division cycle 20 | AGTGCTCCTGGAGAGGGTGGCTGGGTTCCT | | | |
| | | homolog (S. cerevisiae) | | | | |
| | | (CDC20), mRNA | | | | |
| | | [NM_001255] | | | | |
| CDCA3 | NM_031299 | Homo sapiens cell | ACACTACGACAGGGTAAGCGGCCTTCACCC | 8.32E−10 | 2.38825 | 12 |
| | | division cycle associ- | CTAAGTGAAAATGTTAGTGAACTAAAGGAA | | | |
| | | ated 3 (CDCA3), mRNA | | | | |
| | | [NM_031299] | | | | |
| CDCA5 | NM_080668 | Homo sapiens cell | TCACCAGATGATGCAGAGTTGAGATCATCA | 3.15E−08 | 2.0278 | 13 |
| | | division cycle associ- | TTGCAAAGTTCTCTGTTCCTGAGGAACTAA | | | |
| | | ated 5 (CDCA5), mRNA | | | | |
| | | [NM_080668] | | | | |
| CDCA7 | NM_031942 | Homo sapiens cell | ATTTACTTGCATATGTAAACCATTGCTGTG | 4.11E−09 | 2.67162 | 14 |
| | | division cycle associ- | CCATTCAATGTTTGATGCATAATTGGACCT | | | |
| | | ated 7 (CDCA7), trans- | | | | |
| | | cript variant 1, mRNA | | | | |
| | | [NM_031942] | | | | |
| CDCA8 | NM_018101 | Homo sapiens cell | CCCAGGCTTGAAGGCACATGGCTTTCTCAT | 1.03E−08 | 2.13825 | 15 |
| | | division cycle associ- | GTAGGGCTCTCTGTGGTATTTGTTATTATT | | | |
| | | ated 8 (CDCA8), mRNA | | | | |
| | | [NM_018101] | | | | |
| CDT1 | NM_030928 | Homo sapiens chromatin | CACCTTGACTTCAGTATTTCTGACCTCCTA | 1.10E−08 | 2.18541 | 16 |
| | | licensing and DNA | AACTCTAATAAAGTCATGCTTACAGCCACT | | | |
| | | replication factor 1 | | | | |
| | | (CDT1), mRNA | | | | |
| | | [NM_030928] | | | | |
| CENPA | NM_001809 | Homo sapiens centro- | CATGACTAGATCCAATGGATTCTGCGATGC | 1.51E−10 | 2.39079 | 17 |
| | | mere protein A | TGTCTGGACTTTGCTGTCTCTGAACAGTAT | | | |
| | | (CENPA), transcript | | | | |
| | | variant 1, mRNA | | | | |
| | | [NM_001809] | | | | |
| CENPF | NM_016343 | Homo sapiens centro- | AAAGTTTGGAAGCACTGATCACCTGTTAGC | 3.84E−08 | 2.27088 | 18 |
| | | mere protein F, | ATTGCCATTCCTCTACTGCAATGTAAATAG | | | |
| | | 350/400 ka (mitosin) | | | | |
| | | (CENPF), mRNA | | | | |
| | | [NM_016343] | | | | |
| CEP55 | NM_018131 | Homo sapiens centro- | GTAAACCAAAAACTTTTAAATTTCTTCAGG | 2.73E−09 | 2.13814 | 19 |
| | | somal protein 55 kDa | TTTTCTAACATGCTTACCACTGGGCTACTG | | | |
| | | (CEP55), transcript | | | | |
| | | variant 1, mRNA | | | | |
| | | [NM_018131] | | | | |
| CFD | NM_001928 | Homo sapiens comple- | GGCCTGAAGGTCAGGGTCACCCAAGCAACA | 5.06E−10 | −2.78936 | 20 |
| | | ment factor D | AAGTCCCGAGCAATGAAGTCATCCACTCCT | | | |
| | | (adipsin) (CFD), | | | | |
| | | mRNA [NM_001928] | | | | |
| CHAF1B | NM_005441 | Homo sapiens chroma- | CCTGGCATCCTCGTGAAAGTGCACACACTT | 1.30E−08 | 1.91542 | 21 |
| | | tin assembly factor | CATGGAGGGACTCCTTTTCAATAAGAATTA | | | |
| | | 1, subunit B (p60) | | | | |
| | | (CHAF1B), mRNA | | | | |
| | | [NM_005441] | | | | |
| CITED4 | NM_133467 | Homo sapiens Cbp/p300- | ACAGCCCGAACCCGTGGAGCAATGCCCTGT | 8.92E−09 | 2.44312 | 22 |
| | | interacting transac- | CTGGCCTCCAAAACCAAAATAAAACTGGGT | | | |
| | | tivator, with Glu/Asp- | | | | |
| | | rich carboxy-terminal | | | | |
| | | domain, 4 (CITED4), | | | | |
| | | mRNA [NM_133467] | | | | |
| CLEC10A | NM_182906 | Homo sapiens C-type | AGGACTCTTCTCACGACCTCCTCGCAAGAC | 1.34E−10 | −2.77256 | 23 |
| | | lectin domain family | CGCTCTGGGAGAGAAATAAGCACTGGGAGA | | | |
| | | 10, member A (CLEC10A), | | | | |
| | | transcript variant 1, | | | | |
| | | mRNA [NM_182906] | | | | |
| DSC2 | NM_024422 | Homo sapiens desmocollin | CCATCCTTGCAATATTGTTGGGCATAGCAT | 3.79E−10 | 2.30894 | 24 |
| | | 2 (DSC2), transcript | TGCTCTTTTGCATCCTGTTTACGCTGGTCT | | | |
| | | variant Dsc2a, | | | | |
| | | mRNA [NM_024422] | | | | |
| ELF5 | NM_198381 | Homo sapiens E74-like | TCTCAGGTCCAGATGTTAAACGTTTATAAA | 9.64E−10 | 5.25485 | 25 |
| | | factor 5 (ets domain | ACCGGAAATGTCCTAACAACTCTGTAATGG | | | |
| | | transcription factor) | | | | |
| | | (ELF5), transcript | | | | |
| | | variant 1, mRNA | | | | |
| | | [NM_198381] | | | | |
| EXO1 | NM_003686 | Homo sapiens exonu- | AAGCATCCAGAAGAGAAAGCATCATAATGC | 1.72E−08 | 2.21367 | 26 |
| | | clease 1 (EXO1), | CGAGAACAAGCCGGGGTTACAGATCAAACT | | | |
| | | transcript variant | | | | |
| | | 3, mRNA [NM_003686] | | | | |
| FAM64A | NM_019013 | Homo sapiens family | AGGAGGGGTAGCCCTGTTCAAGAGCAATTT | 4.10E−09 | 2.42828 | 27 |
| | | with sequence simi- | CTGCCCTTTGTAAATTATTTAAGAAACCTG | | | |
| | | larity 64, member A | | | | |
| | | (FAM64A), mRNA | | | | |
| | | [NM_019013] | | | | |
| FOXM1 | NM_202002 | Homo sapiens fork- | GGTAGGATGACCTGGGGTTTCAATTGACTT | 6.38E−09 | 2.28481 | 28 |
| | | head box M1 (FOXM1), | CTGTTCCTTGCTTTTAGTTTTGATAGAAGG | | | |
| | | transcript variant | | | | |
| | | 1, mRNA [NM_202002] | | | | |
| FUCA1 | NM_000147 | Homo sapiens fucosi- | TTCTCTGATAACCTACTTGCTTACTCAATG | 5.54E−09 | −1.91098 | 29 |
| | | dase, alpha-L-1, | CCTTTAAGCCAAGTCACCCTGTTGCCTATG | | | |
| | | tissue (FUCA1), | | | | |
| | | mRNA [NM_000147] | | | | |
| GABBR2 | NM_005458 | Homo sapiens gamma- | GAGGAATTTCTCGTACCCCTACTGCATGGT | 1.37E−08 | 4.53168 | 30 |
| | | aminobutyric acid | ATCGATTTTTAATAAATTGTTGCAAATTTG | | | |
| | | (GABA) B receptor, | | | | |
| | | 2 (GABBR2), mRNA | | | | |
| | | [NM_005458] | | | | |
| GIMAP5 | NM_018384 | Homo sapiens GTPase, | TCATTGTTCTAATAATCACCAATTCAGACT | 1.13E−08 | −1.9587 | 31 |
| | | IMAP family member | CAGATCCTCGTGGTCTATGGAGCATGCTGC | | | |
| | | 5 (GIMAP5), mRNA | | | | |
| | | [NM_018384] | | | | |
| GIMAP7 | NM_153236 | Homo sapiens GTPase, | TTTGGGAAGTCAGCCATGAAGCACATGGTC | 2.19E−09 | −2.26543 | 32 |
| | | IMAP family member | ATCTTGTTCACTCGCAAAGAAGAGTTGGAG | | | |
| | | 7 (GIMAP7), mRNA | | | | |
| | | [NM_153236] | | | | |
| GMFG | NM_004877 | Homo sapiens glia | CTCCAAGAAAAGTTGTCTTTCTTTCGTTGA | 7.42E−10 | −1.86818 | 33 |
| | | maturation factor, | TCTCTGGGCTGGGGACTGAATTCCTGATGT | | | |
| | | gamma (GMFG), mRNA | | | | |
| | | [NM_004877] | | | | |
| HDC | NM_002112 | Homo sapiens histi- | CCGAGGGTAGACAGGCAGCTTCTGTGGTTC | 5.10E−11 | −2.85068 | 34 |
| | | dine decarboxylase | AGCTTGTGACATGATATATAACACAGAAAT | | | |
| | | (HDC), mRNA | | | | |
| | | [NM_002112] | | | | |
| HIST1H1A | NM_005325 | Homo sapiens histone | CTGCTAAAGCTAAGGCTGTAAAACCCAAGG | 1.53E−08 | 2.91491 | 35 |
| | | cluster 1, H1a | CGGCCAAGGCTAGGGTGACGAAGCCAAAGA | | | |
| | | (HIST1H1A), mRNA | | | | |
| | | [NM_005325] | | | | |
| HORMAD1 | NM_032132 | Homo sapiens HORMA | AGGTCTAAAGAAAGTCCAGATCTTTCTATT | 3.31E−08 | 3.50544 | 36 |
| | | domain containing 1 | TCTCATTCTCAGGTTGAGCAGTTAGTCAAT | | | |
| | | (HORMAD1), mRNA | | | | |
| | | [NM_032132] | | | | |
| HRASLS | NM_020386 | Homo sapiens HRAS- | TTGGGAGGAGGAAAAGAAACCTGGGGTGAA | 2.16E−09 | 3.25731 | 37 |
| | | like suppressor | TACTTATTTTCAGTGCATCATTACTGTTCC | | | |
| | | (HRASLS), mRNA | | | | |
| | | [NM_020386] | | | | |
| IQGAP3 | NM_178229 | Homo sapiens IQ | ATCTACCCAACTTCCTGTACTGTTGCCCTT | 8.23E−09 | 1.98991 | 38 |
| | | motif containing | CTGATGTTAATAAAAGCAGCTGTTACTCCC | | | |
| | | GTPase activating | | | | |
| | | protein 3 (IQGAP3), | | | | |
| | | mRNA [NM_178229] | | | | |
| ITM2A | NM_004867 | Homo sapiens | CTAGTTGCTGTGGAGGAAATTCGTGATGTT | 2.85E−10 | −2.79709 | 39 |
| | | integral membrane | AGTAACCTTGGCATCTTTATTTACCAACTT | | | |
| | | protein 2A (ITM2A), | | | | |
| | | mRNA [NM_004867] | | | | |
| KCNK5 | NM_003740 | Homo sapiens potassium | CTGTGAAATGTTTTAATGAACCATGTTGTT | 3.44E−08 | 2.54732 | 40 |
| | | channel, subfamily K, | GCTGGTTGTCCTGGCATCGCGCACACTGTA | | | |
| | | member 5 (KCNK5), | | | | |
| | | mRNA [NM_003740] | | | | |
| KLF2 | NM_016270 | Homo sapiens Kruppel- | GAGACAGGTGGGCATTTTTGGGCTACCTGG | 1.15E−08 | −1.86066 | 41 |
| | | like factor 2 (lung) | TTCGTTTTTATAAGATTTTGCTGGGTTGGT | | | |
| | | (KLF2), mRNA | | | | |
| | | [NM_016270] | | | | |
| KRTCAP3 | NM_173853 | Homo sapiens kera- | GCTAGAGGAAATGACAGAGCTCGAATCTCC | 5.21E−09 | 2.80703 | 42 |
| | | tinocyte associated | TAAATGTAAAAGGCAGGAAAATGAGCAGCT | | | |
| | | protein 3 (KRTCAP3), | | | | |
| | | mRNA [NM_173853] | | | | |
| LILRB5 | NM_006840 | Homo sapiens leukocyte | CTAGATTCTGCAGTCAAAGATGACTAATAT | 2.39E−08 | −2.3384 | 43 |
| | | immunoglobulin-like | CCTTGCATTTTTGAAATGAAGCCACAGACT | | | |
| | | receptor, subfamily B | | | | |
| | | (with TM and ITIM | | | | |
| | | domains), member 5 | | | | |
| | | (LILRB5), transcript | | | | |
| | | variant 2, mRNA | | | | |
| | | [NM_006840] | | | | |
| LRMP | NM_006152 | Homo sapiens lymphoid- | AGGTTCTCAGAATGACCGTAAGATAGCTTA | 4.95E−10 | −2.20204 | 44 |
| | | restricted membrane | CATTTCCTCTTTTTGCCTTTATCTCCCCAA | | | |
| | | protein (LRMP), mRNA | | | | |
| | | [NM_006152] | | | | |
| MCM10 | NM_182751 | Homo sapiens mini- | TGCTCTTACATTATTGTGGAGCCCTGTGAT | 6.82E−09 | 2.27218 | 45 |
| | | chromosome maintenance | AGAAATATGTAAAATCTCATATTATTTTTT | | | |
| | | complex component 10 | | | | |
| | | (MCM10), transcript | | | | |
| | | variant 1, mRNA | | | | |
| | | [NM_182751] | | | | |
| MCM2 | NM_004526 | Homo sapiens mini- | TTTGGGTGGGATGCCTTGCCAGTGTGTCTT | 4.00E−09 | 1.89845 | 46 |
| | | chromosome maintenance | ACTTGGTTGCTGAACATCTTGCCACCTCCG | | | |
| | | complex component 2 | | | | |
| | | (MCM2), mRNA | | | | |
| | | [NM_004526] | | | | |
| MELK | NM_014791 | Homo sapiens maternal | GGAAAGTGACAATGCAATTTGAATTAGAAG | 2.91E−08 | 2.3082 | 47 |
| | | embryonic leucine zipper | TGTGCCAGCTTCAAAAACCCGATGTGGTGG | | | |
| | | kinase (MELK), mRNA | | | | |
| | | [NM_014791] | | | | |
| MFAP4 | NM_002404 | Homo sapiens micro- | AAATTACACCTGGAGTCAGGTGCAGAAGGG | 3.10E−09 | −3.17716 | 48 |
| | | fibrillar-associated | AACCTTGTATTTCACAGGCCTCATTTTGAT | | | |
| | | protein 4 (MFAP4), | | | | |
| | | mRNA [NM_002404] | | | | |
| MIAT | NR_003491 | Homo sapiens myocardial | TGGCTGAGATGATACCCGACCCTCTAGGGA | 1.89E−08 | −2.35646 | 49 |
| | | infarction associated | | | | |
| | | transcript (non-protein | AATTCTTAGAGTAACTTCTAGGAAATGTCA | | | |
| | | coding) (MIAT), non- | | | | |
| | | coding RNA [NR_003491] | | | | |
| NRTN | NM_004558 | Homo sapiens neurturin | TGGACGCGCACAGCCGCTACCACACGGTGC | 3.35E−14 | 3.67281 | 50 |
| | | (NRTN), mRNA [NM_004558] | ACGAGCTGTCGGCGCGCGAGTGCGCCTGCG | | | |
| OGN | NM_033014 | Homo sapiens osteoglycin | AACTAATGATCACAGCTATTATACTACTTT | 8.77E−09 | −3.70339 | 51 |
| | | (OGN), transcript variant | CTCGTTATTTTGTGTGCATGCCTCATTTCC | | | |
| | | 1, mRNA [NM_033014] | | | | |
| PADI2 | NM_007365 | Homo sapiens peptidyl | AGAGCTGAAAACACCAAGTGCCTATTTGAG | 6.23E−09 | 2.83004 | 52 |
| | | arginine deiminase, | GGTGTCTGTCTGGAGACTTAGAGTTTGTCA | | | |
| | | type II (PADI2), mRNA | | | | |
| | | [NM_007365] | | | | |
| PHGDH | NM_006623 | Homo sapiens phospho- | TTGGTCCAAGGCACTACACCTGTACTGCAG | 1.07E−10 | 2.95348 | 53 |
| | | glycerate dehydrogenase | GGGCTCAATGGAGCTGTCTTCAGGCCAGAA | | | |
| | | (PHGDH), mRNA | | | | |
| | | [NM_006623] | | | | |
| PLCB4 | NM_000933 | Homo sapiens phospho- | CCTTATCTGTAAAACAGTGGAGTTAGACTA | 2.00E−08 | 2.1783 | 54 |
| | | lipase C, beta 4 | CATATCTTTTGGCACTAACATCTCATGAAA | | | |
| | | (PLCB4), transcript | | | | |
| | | variant 1, mRNA | | | | |
| | | [NM_000933] | | | | |
| PLEKHB1 | NM_021200 | Homo sapiens | TAAAGCTCCCCTGTAAATGGGGGCTCCATT | 3.39E−11 | 3.30942 | 55 |
| | | pleckstrin homology | AGTTCTGCTGCCGAGACTAATAAAGATTTG | | | |
| | | domain containing, | | | | |
| | | family B (evectins) | | | | |
| | | member 1 (PLEKHB1), | | | | |
| | | transcript variant | | | | |
| | | 1, mRNA [NM_021200] | | | | |
| PROM1 | NM_001145850 | Homo sapiens | TTTTTGCGGTAAAACTGGCTAAGTACTATC | 6.77E−09 | 4.6813 | 56 |
| | | prominin 1 (PROM1), | GTCGAATGGATTCGGAGGACGTGTACGATG | | | |
| | | transcript variant | | | | |
| | | 6, mRNA [NM_001145850] | | | | |
| PSAT1 | NM_058179 | Homo sapiens phospho- | TACCATTCTTTCCATAGGTAGAAGAGAAAG | 2.59E−09 | 2.92479 | 57 |
| | | serine aminotrans- | TTGATTGGTTGGTTGTTTTTCAATTATGCC | | | |
| | | ferase 1 (PSAT1), | | | | |
| | | transcript variant | | | | |
| | | 1, mRNA [NM_058179] | | | | |
| PTCRA | NM_138296 | Homo sapiens pre T- | ACAGGGGCATTTAGGGAGCAGATGACTGAG | 2.13E−08 | −2.02765 | 58 |
| | | cell antigen receptor | AACATTAAAAAAGAACTTAAATGACACAGC | | | |
| | | alpha (PTCRA), | | | | |
| | | mRNA [NM_138296] | | | | |
| PTGDS | NM_000954 | Homo sapiens prosta- | CAAAGCAACCCTGCCCACTCAGGCTTCATC | 2.88E−09 | −3.30008 | 59 |
| | | glandin D2 synthase 21 | CTGCACAATAAACTCCGGAAGCAAGTCAGT | | | |
| | | kDa (brain) (PTGDS), | | | | |
| | | mRNA [NM_000954] | | | | |
| RAD51AP1 | NM_006479 | Homo sapiens RAD51 | GGTTGGGAGAATCACAGCTTTACAAGGGTG | 5.08E−09 | 2.09804 | 60 |
| | | associated protein 1 | TTTATATTTGATTTGTGTTTATATTTGAGG | | | |
| | | (RAD51AP1), transcript | | | | |
| | | variant 2, mRNA | | | | |
| | | [NM_006479] | | | | |
| ROPN1 | NM_017578 | Homo sapiens ropporin, | GAATGACTTTACCCAAAACCCCAGGGTTCA | 2.93E−10 | 7.63253 | 61 |
| | | rhophilin associated | GCTGGAGTAAAAGCACAATTTTGGCAATTT | | | |
| | | protein 1 (ROPN1), | | | | |
| | | mRNA [NM_017578] | | | | |
| ROPN1B | NM_001012337 | Homo sapiens ropporin, | TGGCAATTTTAAAGGAAGATACAGAGGTGA | 5.01E−10 | 6.13033 | 62 |
| | | rhophilin associated | TTGTACTTCAGAATGATAAACCCATATACC | | | |
| | | protein 1B (ROPN1B), | | | | |
| | | mRNA [NM_001012337] | | | | |
| RPL39L | NM_052969 | Homo sapiens ribosomal | GAGAGAAGCAAGCATCTTTGCCTCTTTGGA | 1.86E−09 | 1.83988 | 63 |
| | | protein L39-like | | | | |
| | | (RPL39L), mRNA | GTAGGAAATTCAGACTTGAAAAAGTGGTGT | | | |
| | | [NM_052969] | | | | |
| SCML4 | NM_198081 | Homo sapiens sex comb | CATTTTGCATTAAACTTTAAGCAGGACAGA | 2.20E−08 | −2.67377 | 64 |
| | | on midleg-like 4 | TTGCTGAAGCCATGATATTTAAGGTTTGAC | | | |
| | | (Drosophila) (SCML4), | | | | |
| | | mRNA [NM_198081] | | | | |
| SLC40A1 | NM_014585 | Homo sapiens solute | CTCATGTTATCATCATTAGTGATCTGTGTT | 3.12E−09 | −2.86082 | 65 |
| | | carrier family 40 | GTAGAACATGAGGGTGTAAGCCTTCAGCCT | | | |
| | | (iron-regulated | | | | |
| | | transporter), member | | | | |
| | | 1 (SLC40A1), mRNA | | | | |
| | | [NM_014585] | | | | |
| SLC7A8 | NM_182728 | Homo sapiens solute | TTTTTTGTAAAGTTGATGCCTTACTTTTTG | 9.52E−09 | −2.29559 | 66 |
| | | carrier family 7 | GATAAATATTTTTGAAGCTGGTATTTCTAT | | | |
| | | (cationic amino acid | | | | |
| | | transporter, y+ system), | | | | |
| | | member 8 (SLC7A8), | | | | |
| | | transcript variant 2, | | | | |
| | | mRNA [NM_182728] | | | | |
| SUV39H2 | NM_024670 | Homo sapiens suppres- | ATTTGCCAAATGTATTACCGATGCCTCTGA | 2.32E−09 | 1.87415 | 67 |
| | | sor of variegation | AAAGGGGGTCACTGGGTCTCATAGACTGAT | | | |
| | | 3-9 homolog 2 | | | | |
| | | (Drosophila) (SUV39H2), | | | | |
| | | mRNA [NM_024670] | | | | |
| TBC1D10C | NM_198517 | Homo sapiens TBC1 | GGAAGGGGTTGGCTGAGTCAAGGGACCCCA | 6.20E−09 | −2.30803 | 68 |
| | | domain family, member | GAGGGCACCAGGAATAAAATCTTCTTGAAC | | | |
| | | 10C (TBC1D10C), | | | | |
| | | mRNA [NM_198517] | | | | |
| TBC1D9 | NM_015130 | Homo sapiens TBC1 | AAACATCCGGATGATGGGCAAGCCCCTCAC | 1.92E−08 | −2.16865 | 69 |
| | | domain family, member | CTCGGCCAGTGACTATGAAATCTCGGCCAT | | | |
| | | 9 (with GRAM domain) | | | | |
| | | (TBC1D9), mRNA | | | | |
| | | [NM_015130] | | | | |
| TFCP2L1 | NM_014553 | Homo sapiens trans- | GATGGTGGGCTAAATTTTAATTCTCAAAAG | 2.97E−08 | 3.56367 | 70 |
| | | cription factor CP2- | TGTAGGAGGCTAATATTGTCTTCTAAGTTC | | | |
| | | like 1 (TFCP2L1), | | | | |
| | | mRNA [NM_014553] | | | | |
| TMEM38A | NM_024074 | Homo sapiens trans- | TTCACAGAATCCTGGCAGCAGCTCCAGTCA | 1.97E−10 | 2.2764 | 71 |
| | | membrane protein 38A | AGAATGTCACTGGTTGGCATGATATTCTTA | | | |
| | | (TMEM38A), mRNA | | | | |
| | | [NM_024074] | | | | |
| TPX2 | NM_012112 | Homo sapiens TPX2, | AGAGAACCCATTTCTCCAGACTTTTACCTA | 1.29E−09 | 2.28201 | 72 |
| | | microtubule-associated, | CCCGTGCCTGAGAAAGCATACTTGACAACT | | | |
| | | homolog (Xenopus laevis) | | | | |
| | | (TPX2), mRNA | | | | |
| | | [NM_012112] | | | | |
| TRIM2 | NM_015271 | Homo sapiens tripartite | GATGCTTAAAAACTTTCTAAAGATGAATTG | 5.65E−09 | 2.3576 | 73 |
| | | motif-containing 2 | TGTGGCAGTGATTGGTCTGTTTGTGGAGAA | | | |
| | | (TRIM2), transcript | | | | |
| | | variant 1, mRNA | | | | |
| | | [NM_015271] | | | | |
| TTK | NM_003318 | Homo sapiens TTK protein | TGTTTGGTCCTTAGGATGTATTTTGTACTA | 5.26E−11 | 2.39315 | 74 |
| | | kinase (TTK), transcript | TATGACTTACGGGAAAACACCATTTCAGCA | | | |
| | | variant 1, mRNA | | | | |
| | | [NM_003318] | | | | |
| TTYH1 | NM_020659 | Homo sapiens tweety | GGCTCTGACCCCCTGATCTCAACTCGTGGC | 2.16E−08 | 4.69134 | 75 |
| | | homolog 1 (Drosophila) | ACTAACTTGGAAAAGGGTTGATTTAAAATA | | | |
| | | (TTYH1), transcript | | | | |
| | | variant 1, mRNA | | | | |
| | | [NM_020659] | | | | |
| UGT8 | NM_003360 | Homo sapiens UDP | TGCCGCTGTCCATCAGATCTCCTTTTGTCA | 1.12E−08 | 2.48001 | 76 |
| | | glycosyltransferase | GTATTTTTTACTGGATATTGCCTTTGTGCT | | | |
| | | 8 (UGT8), transcript | | | | |
| | | variant 2, mRNA | | | | |
| | | [NM_003360] | | | | |
| VGLL1 | NM_016267 | Homo sapiens vestigial | AGACACGGCAGCAAGACATCCCTGCATATT | 1.61E−10 | 5.4559 | 77 |
| | | like 1 (Drosophila) | GTTCCAGATAAAAATGAAAGCTGCTCACAC | | | |
| | | (VGLL1), mRNA | | | | |
| | | [NM_016267] | | | | |

TABLE 2

| | | SEQ |
|---|---|---|
| | | ID |
| hgnc_symbol | Sequence | NO |
| ABCA6 | ATTAGTAAAGTCACCCAAAGAGTCAGGCACTGGGTATTGTGGAAATAAAACTATATAAAC | 1 |
| ACTR3B | ATAGAAGATGATGGTTTGTTGTCGGTGAGTGTTGGATGAAATACTTCCTTGCACCATTGT | 2 |
| ACTR3B | CCCGGAAGTGGATCAAACAGTACACGGGTATCAATGCGATCAACCAGAAGAAGTTTGTTA | 78 |
| ACTR3B | TAGAGAAAACAACATTAGAAAATGGCGCAAAATCGTTAGGTCCCAGGAGAGAATGTGGGG | 79 |
| ACTR3B | ATAGAAGATGATGGTTTGTTGTCGGTGAGTGTTGGATGAAATACTTCCTTGCACCATTGT | 80 |
| ADRB2 | CTCTTATTTGCTCACACGGGGTATTTTAGGCAGGGATTTGAGGAGCAGCTTCAGTTGTTT | 3 |
| AMICA1 | CTCCTGTGGGCAGGGTTCTTAGTGGATGAGTTACTGGGAAGAATCAGAGATAAAAACCAA | 4 |
| ATP8A1 | CTATGCAGTGTTATGTGTCATTGGCCTTTTGTGAATGTGCATGTTTTAAACTGCAAATTT | 5 |
| AURKB | GTCTGTGTATGTATAGGGGAAAGAAGGGATCCCTAACTGTTCCCTTATCTGTTTTCTACC | 6 |
| AURKB | AATAGCAGTGGGACACCCGACATCTTAACGCGGCACTTCACAATTGATGACTTTGAGATT | 81 |
| B3GNT5 | TGGTGCTCCAGTGTAGGGCTATCTTTTTAAAAAATGTCAACAAAGGGAAAATAAACTATC | 7 |
| B3GNT5 | AAATGTCAACAAAGGGAAAATAAACTATCAGCTTGGATGGTCACTTGAATAGAAGATGGT | 82 |
| BASP1 | TTCAGTCAACTTTACCAAGAAGTCCTGGATTTCCAAGATCCGCGTCTGAAAGTGCAGTAC | 8 |
| BASP1 | TCAATGCCAATCCTCCATTCTTCCTCTCCAGATATTTTTGGGAGTGACAAACATTCTCTC | 83 |
| C10orf35 | GGAGCAGGACTTGGGCTTAGGGCAGGTGGAAAAAATTCCAGACTTTTTTAGCACTGTTTT | 9 |
| CCNA2 | AAGTTTGATAGATGCTGACCCATACCTCAAGTATTTGCCATCAGTTATTGCTGGAGCTGC | 10 |
| CCNA2 | AAGTTTGATAGATGCTGACCCATACCTCAAGTATTTGCCATCAGTTATTGCTGGAGCTGC | 84 |
| CDC20 | ATCCACCAAGGCATCCGCTGAAGACCAACCCATCACCTCAGTTGTTTTTTATTTTTCTAA | 11 |
| CDC20 | GGTAATGATAACTTGGTCAATGTGTGGCCTAGTGCTCCTGGAGAGGGTGGCTGGGTTCCT | 85 |
| CDCA3 | ACACTACGACAGGGTAAGCGGCCTTCACCCCTAAGTGAAAATGTTAGTGAACTAAAGGAA | 12 |
| CDCA3 | AGGAATGGCTTGTTTTCTTAGACTCCTCCTCAGCTACCAAACTGGGACTCACAGCTTTAT | 86 |
| CDCA5 | TCACCAGATGATGCAGAGTTGAGATCATCATTGCAAAGTTCTCTGTTCCTGAGGAACTAA | 13 |
| CDCA7 | GCTGTGCCATTCAATGTTTGATGCATAATTGGACCTTGAATCGATAAGTGTAAATACAGC | 14 |
| CDCA7 | GCATAATATCTGGAAAATTTGCTGCCTGCCTTCTACTTCTCAAATCTTTCTTGTAAAAGT | 87 |
| CDCA7 | ATTTACTTGCATATGTAAACCATTGCTGTGCCATTCAATGTTTGATGCATAATTGGACCT | 88 |
| CDCA8 | CCCAGGCTTGAAGGCACATGGCTTTCTCATGTAGGGCTCTCTGTGGTATTTGTTATTATT | 15 |
| CDCA8 | CCCAGGCTTGAAGGCACATGGCTTTCTCATGTAGGGCTCTCTGTGGTATTTGTTATTATT | 89 |
| CDT1 | CACCTTGACTTCAGTATTTCTGACCTCCTAAACTCTAATAAAGTCATGCTTACAGCCACT | 16 |
| CENPA | TAGTTTGTGAGTTACTCATGTGACTATTTGAGGATTTTGAAAACATCAGATTTGCTGTGG | 17 |
| CENPA | GGGGATGAATAGAAAACCTGTAAGCTTTGATGTTCTGGTTACTTCTAGTAAATTCCTGTC | 90 |
| CENPA | CATGACTAGATCCAATGGATTCTGCGATGCTGTCTGGACTTTGCTGTCTCTGAACAGTAT | 91 |
| CENPF | CAGGACTTCTCTTTAGTCAGGGCATGCTTTATTAGTGAGGAGAAAACAATTCCTTAGAAG | 18 |
| CENPF | GCTGGAGATAGACCTTTTAAAGTCTAGTAAAGAAGAGCTCAATAATTCATTGAAAGCTAC | 92 |
| CENPF | AAAGTTTGGAAGCACTGATCACCTGTTAGCATTGCCATTCCTCTACTGCAATGTAAATAG | 93 |
| CEP55 | GACCGTCAACATGTGCAGCATCAATTGCATGTAATTCTTAAGGAGCTCCGAAAAGCAAGA | 19 |
| CEP55 | GTAAACCAAAAACTTTTAAATTTCTTCAGGTTTTCTAACATGCTTACCACTGGGCTACTG | 94 |
| CEP55 | GTAAACCAAAAACTTTTAAATTTCTTCAGGTTTTCTAACATGCTTACCACTGGGCTACTG | 95 |
| CFD | GGCCTGAAGGTCAGGGTCACCCAAGCAACAAAGTCCCGAGCAATGAAGTCATCCACTCCT | 20 |
| CHAF1B | CCTGGCATCCTCGTGAAAGTGCACACACTTCATGGAGGGACTCCTTTTCAATAAGAATTA | 21 |
| CITED4 | ACAGCCCGAACCCGTGGAGCAATGCCCTGTCTGGCCTCCAAAACCAAAATAAAACTGGGT | 22 |
| CLEC10A | AGGACTCTTCTCACGACCTCCTCGCAAGACCGCTCTGGGAGAGAAATAAGCACTGGGAGA | 23 |
| DSC2 | CCATCCTTGCAATATTGTTGGGCATAGCATTGCTCTTTTGCATCCTGTTTACGCTGGTCT | 24 |
| DSC2 | CAAATTTAGGACACTAGCAGAAGCATGCATGAAGAGATGAGTGTGTTCTAATAAGTCTCT | 96 |
| ELF5 | TCTCAGGTCCAGATGTTAAACGTTTATAAAACCGGAAATGTCCTAACAACTCTGTAATGG | 25 |
| ELF5 | TCTCAGGTCCAGATGTTAAACGTTTATAAAACCGGAAATGTCCTAACAACTCTGTAATGG | 97 |
| EXO1 | AAGCATCCAGAAGAGAAAGCATCATAATGCCGAGAACAAGCCGGGGTTACAGATCAAACT | 26 |
| EXO1 | AAGCATCCAGAAGAGAAAGCATCATAATGCCGAGAACAAGCCGGGGTTACAGATCAAACT | 98 |
| FAM64A | AGGAGGGGTAGCCCTGTTCAAGAGCAATTTCTGCCCTTTGTAAATTATTTAAGAAACCTG | 27 |
| FAM64A | AAGAAACCAGCATGTGACTTTCCTAGATAACACTGCTTTCTCATAATAAAGACTATTTGC | 99 |
| FAM64A | AAACAGCATTATGGAGTTAAAAGATTTTTACAACTGGGTCTTGATTTTGATGTGAGCTGG | 100 |
| FAM64A | GAATTCAGCATCTCCAGAAGCTGTCCCAAGAGCTAGATGAAGCCATTATGGCGGAAGAGA | 101 |
| FOXM1 | GGTAGGATGACCTGGGGTTTCAATTGACTTCTGTTCCTTGCTTTTAGTTTTGATAGAAGG | 28 |
| FOXM1 | GGTAGGATGACCTGGGGTTTCAATTGACTTCTGTTCCTTGCTTTTAGTTTTGATAGAAGG | 102 |
| FUCA1 | TTCTCTGATAACCTACTTGCTTACTCAATGCCTTTAAGCCAAGTCACCCTGTTGCCTATG | 29 |
| GABBR2 | GAGGAATTTCTCGTACCCCTACTGCATGGTATCGATTTTTAATAAATTGTTGCAAATTTG | 30 |
| GIMAP5 | TCATTGTTCTAATAATCACCAATTCAGACTCAGATCCTCGTGGTCTATGGAGCATGCTGC | 31 |
| GIMAP7 | TTTGGGAAGTCAGCCATGAAGCACATGGTCATCTTGTTCACTCGCAAAGAAGAGTTGGAG | 32 |
| GIMAP7 | TTTGGGAAGTCAGCCATGAAGCACATGGTCATCTTGTTCACTCGCAAAGAAGAGTTGGAG | 103 |
| GMFG | CTCCAAGAAAAGTTGTCTTTCTTTCGTTGATCTCTGGGCTGGGGACTGAATTCCTGATGT | 33 |
| HDC | CCGAGGGTAGACAGGCAGCTTCTGTGGTTCAGCTTGTGACATGATATATAACACAGAAAT | 34 |
| HIST1H1A | CTGCTAAAGCTAAGGCTGTAAAACCCAAGGCGGCCAAGGCTAGGGTGACGAAGCCAAAGA | 35 |
| HORMAD1 | AGGTCTAAAGAAAGTCCAGATCTTTCTATTTCTCATTCTCAGGTTGAGCAGTTAGTCAAT | 36 |
| HORMAD1 | CCCAGATTACCAGCCTCCCGGTTTTAAGGATGGTGATTGTGAAGGAGTTATATTTGAAGG | 104 |
| HRASLS | GTGGCCTATAACTTACTTGTCAACAACTGTGAACATTTTGTGACATTGCTTCGCTATGGA | 37 |
| HRASLS | TTGGGAGGAGGAAAAGAAACCTGGGGTGAATACTTATTTTCAGTGCATCATTACTGTTCC | 105 |
| IQGAP3 | ATCTACCCAACTTCCTGTACTGTTGCCCTTCTGATGTTAATAAAAGCAGCTGTTACTCCC | 38 |
| ITM2A | CTAGTTGCTGTGGAGGAAATTCGTGATGTTAGTAACCTTGGCATCTTTATTTACCAACTT | 39 |
| KCNK5 | CTGTCTCCAGGTAGGTGGACCAGAGAACTTGAGCGAAGCTCAAGCCTTCTCAACTCAAGG | 40 |
| KCNK5 | CTGTGAAATGTTTTAATGAACCATGTTGTTGCTGGTTGTCCTGGCATCGCGCACACTGTA | 106 |
| KCNK5 | CTGTGAAATGTTTTAATGAACCATGTTGTTGCTGGTTGTCCTGGCATCGCGCACACTGTA | 107 |
| KLF2 | GAGACAGGTGGGCATTTTTGGGCTACCTGGTTCGTTTTTATAAGATTTTGCTGGGTTGGT | 41 |
| KRTCAP3 | GCTAGAGGAAATGACAGAGCTCGAATCTCCTAAATGTAAAAGGCAGGAAAATGAGCAGCT | 42 |
| LILRB5 | CTAGATTCTGCAGTCAAAGATGACTAATATCCTTGCATTTTTGAAATGAAGCCACAGACT | 43 |
| LRMP | AGGTTCTCAGAATGACCGTAAGATAGCTTACATTTCCTCTTTTTGCCTTTATCTCCCCAA | 44 |
| MCM10 | CCTCCTGTGACTCTGGAAAGCAAAGGATTGGCTGTGTATTGTCCATTGATTCCTGATTGA | 45 |
| MCM10 | TGCTCTTACATTATTGTGGAGCCCTGTGATAGAAATATGTAAAATCTCATATTATTTTTT | 108 |
| MCM2 | TTTGGGTGGGATGCCTTGCCAGTGTGTCTTACTTGGTTGCTGAACATCTTGCCACCTCCG | 46 |
| MCM2 | TTTGGGTGGGATGCCTTGCCAGTGTGTCTTACTTGGTTGCTGAACATCTTGCCACCTCCG | 109 |
| MELK | GATACAGCCTACATAAAGACTGTTATGATCGCTTTGATTTTAAAGTTCATTGGAACTACC | 47 |
| MELK | GGAAAGTGACAATGCAATTTGAATTAGAAGTGTGCCAGCTTCAAAAACCCGATGTGGTGG | 110 |
| MFAP4 | AAATTACACCTGGAGTCAGGTGCAGAAGGGAACCTTGTATTTCACAGGCCTCATTTTGAT | 48 |
| MIAT | CAACAAAGGAGCGTCACTTGGATTTTTGTTTTCATCCATGAATGTAGCTGCTTCTGTGTA | 49 |
| MIAT | TGGCTGAGATGATACCCGACCCTCTAGGGAAATTCTTAGAGTAACTTCTAGGAAATGTCA | 111 |
| NRTN | TGGACGCGCACAGCCGCTACCACACGGTGCACGAGCTGTCGGCGCGCGAGTGCGCCTGCG | 50 |
| OGN | GGTACATGTTCCAAAAACTTTGAAAAGCTAAATGTTTCCCATGATCGCTCATTCTTCTTT | 51 |
| OGN | AACTAATGATCACAGCTATTATACTACTTTCTCGTTATTTTGTGTGCATGCCTCATTTCC | 112 |
| PADI2 | TCTAAGGCTTTCCCCAATGATGTCGGTAATTTCTGATGTTTCTGAAGTTCCCAGGACTCA | 52 |
| PADI2 | GCTGAAGGTCTGCTTCCAGTACCTAAACCGAGGCGATCGCTGGATCCAGGATGAAATTGA | 113 |
| PADI2 | AGAGCTGAAAACACCAAGTGCCTATTTGAGGGTGTCTGTCTGGAGACTTAGAGTTTGTCA | 114 |
| PHGDH | ACCCACCCACTGTGATCAATAGGGAGAGAAAATCCACATTCTTGGGCTGAACGCGGGCCT | 53 |
| PHGDH | TTGGTCCAAGGCACTACACCTGTACTGCAGGGGCTCAATGGAGCTGTCTTCAGGCCAGAA | 115 |
| PLCB4 | CCTTATCTGTAAAACAGTGGAGTTAGACTACATATCTTTTGGCACTAACATCTCATGAAA | 54 |
| PLCB4 | ACAGATCTAGTGAACATTAGTTTTACCTACATGGTGGCTGAAAATCCAGAAGTAACTAAG | 116 |
| PLEKHB1 | TAAAGCTCCCCTGTAAATGGGGGCTCCATTAGTTCTGCTGCCGAGACTAATAAAGATTTG | 55 |
| PROM1 | TGGGGTGTTTGTTCCCATTGGATGCATTTCTATCAAAACTCTATCAAATGTGATGGCTAG | 56 |
| PROM1 | TTTTTGCGGTAAAACTGGCTAAGTACTATCGTCGAATGGATTCGGAGGACGTGTACGATG | 117 |
| PSAT1 | TACCATTCTTTCCATAGGTAGAAGAGAAAGTTGATTGGTTGGTTGTTTTTCAATTATGCC | 57 |
| PSAT1 | GATGCATCAGCTATGAACACATCCTAACCAGGATATACTCTGTTCTTGAACAACATACAA | 118 |
| PTCRA | ACAGGGGCATTTAGGGAGCAGATGACTGAGAACATTAAAAAAGAACTTAAATGACACAGC | 58 |
| PTGDS | CAAAGCAACCCTGCCCACTCAGGCTTCATCCTGCACAATAAACTCCGGAAGCAAGTCAGT | 59 |
| RAD51AP1 | GGTTGGGAGAATCACAGCTTTACAAGGGTGTTTATATTTGATTTGTGTTTATATTTGAGG | 60 |
| ROPN1 | GAATGACTTTACCCAAAACCCCAGGGTTCAGCTGGAGTAAAAGCACAATTTTGGCAATTT | 61 |
| ROPN1 | GAATGACTTTACCCAAAACCCCAGGGTTCAGCTGGAGTAAAAGCACAATTTTGGCAATTT | 119 |
| ROPN1B | TGGCAATTTTAAAGGAAGATACAGAGGTGATTGTACTTCAGAATGATAAACCCATATACC | 62 |
| RPL39L | GAGAGAAGCAAGCATCTTTGCCTCTTTGGAGTAGGAAATTCAGACTTGAAAAAGTGGTGT | 63 |
| SCML4 | TCACCTTGCACTGTCTGGAAAACTTGAATTATTTTACGCCGTGAAAGAAAAAGGAAAAAA | 64 |
| SCML4 | CATTTTGCATTAAACTTTAAGCAGGACAGATTGCTGAAGCCATGATATTTAAGGTTTGAC | 120 |
| SLC40A1 | CTCATGTTATCATCATTAGTGATCTGTGTTGTAGAACATGAGGGTGTAAGCCTTCAGCCT | 65 |
| SLC40A1 | CTCATGTTATCATCATTAGTGATCTGTGTTGTAGAACATGAGGGTGTAAGCCTTCAGCCT | 121 |
| SLC7A8 | TTTTTTGTAAAGTTGATGCCTTACTTTTTGGATAAATATTTTTGAAGCTGGTATTTCTAT | 66 |
| SLC7A8 | TTTTTTGTAAAGTTGATGCCTTACTTTTTGGATAAATATTTTTGAAGCTGGTATTTCTAT | 122 |
| SLC7A8 | CCTGTCTATTTCCTGGGTGTTTACTGGCAACACAAGCCCAAGTGTTTCAGTGACTTCATT | 123 |
| SUV39H2 | ATTTGCCAAATGTATTACCGATGCCTCTGAAAAGGGGGTCACTGGGTCTCATAGACTGAT | 67 |
| TBC1D10C | GGAAGGGGTTGGCTGAGTCAAGGGACCCCAGAGGGCACCAGGAATAAAATCTTCTTGAAC | 68 |
| TBC1D9 | AAACATCCGGATGATGGGCAAGCCCCTCACCTCGGCCAGTGACTATGAAATCTCGGCCAT | 69 |
| TBC1D9 | CTGGATGTTTAGCTTCTTACTGCAAAAACATAAGTAAAACAGTCAACTTTACCATTTCCG | 124 |
| TBC1D9 | TGTCACAGAGAATCTGAAAGTAGCAGCAAAGACAGAGGGCTCATGACAGGTTTTTGCTTT | 125 |
| TFCP2L1 | GATGGTGGGCTAAATTTTAATTCTCAAAAGTGTAGGAGGCTAATATTGTCTTCTAAGTTC | 70 |
| TFCP2L1 | GATGGTGGGCTAAATTTTAATTCTCAAAAGTGTAGGAGGCTAATATTGTCTTCTAAGTTC | 126 |
| TMEM38A | TTCACAGAATCCTGGCAGCAGCTCCAGTCAAGAATGTCACTGGTTGGCATGATATTCTTA | 71 |
| TPX2 | AGAGAACCCATTTCTCCAGACTTTTACCTACCCGTGCCTGAGAAAGCATACTTGACAACT | 72 |
| TPX2 | AGAGAACCCATTTCTCCAGACTTTTACCTACCCGTGCCTGAGAAAGCATACTTGACAACT | 127 |
| TRIM2 | GATGCTTAAAAACTTTCTAAAGATGAATTGTGTGGCAGTGATTGGTCTGTTTGTGGAGAA | 73 |
| TRIM2 | GATGCTTAAAAACTTTCTAAAGATGAATTGTGTGGCAGTGATTGGTCTGTTTGTGGAGAA | 128 |
| TTK | TGTTTGGTCCTTAGGATGTATTTTGTACTATATGACTTACGGGAAAACACCATTTCAGCA | 74 |
| TTK | TGTTTGGTCCTTAGGATGTATTTTGTACTATATGACTTACGGGAAAACACCATTTCAGCA | 129 |
| TTYH1 | GGCTCTGACCCCCTGATCTCAACTCGTGGCACTAACTTGGAAAAGGGTTGATTTAAAATA | 75 |
| UGT8 | TGCCGCTGTCCATCAGATCTCCTTTTGTCAGTATTTTTTACTGGATATTGCCTTTGTGCT | 76 |
| VGLL1 | AGACACGGCAGCAAGACATCCCTGCATATTGTTCCAGATAAAAATGAAAGCTGCTCACAC | 77 |

TABLE 3
3A Top 2 genes in training data set

| | | |
|---|---|---|
| Real\Predicted | 0 | 1 |
| 0 | 58 | 9 |
| 1 | 12 | 49 |

| | |
|---|---|
| Positive Outcome | 1 |
| Negative Outcome | Outcome(s) other than 1 |
| Actual Positive (P): | 61 |
| Actual Negative (N): | 67 |
| Predictived Positive (P′): | 58 |
| Predictived Negative (N′): | 70 |
| True Positive (TP): | 49 |
| False Positive (FP): | 9 |
| False Negative (FN): | 12 |
| True Negative (TN): | 58 |
| Sensitivity (TP/(TP + FN)): | 0.8033 |
| Specificity (TN/(FP + TN)): | 0.8657 |
| Positive Predictive Value (TP/(TP + FP)): | 0.8448 |
| Negative Predictive Value (TN/(FN + TN)): | 0.8286 |
| Matthews Correlation Coefficient | 0.6712 |
| ((TP*TN − FP*FN)/sqrt(P*N*P′*N′)): | |
| Area Under Curve (((TP/(TP + FN)) + | 0.8345 |
| (TN/FP + TN))*0.5): | |

3B Top 2 genes in validation data set

| | | |
|---|---|---|
| Real\Predicted | 0 | 1 |
| 0 | 0 | 28 |
| 1 | 0 | 25 |

| | |
|---|---|
| Positive Outcome | 1 |
| Negative Outcome | Outcome(s) other than 1 |
| Actual Positive (P): | 25 |
| Actual Negative (N): | 28 |
| Predictived Positive (P′): | 53 |
| Predictived Negative (N′): | 0 |
| True Positive (TP): | 25 |
| False Positive (FP): | 28 |
| False Negative (FN): | 0 |
| True Negative (TN): | 0 |
| Sensitivity (TP/(TP + FN)): | 1.0000 |
| Specificity (TN/(FP + TN)): | 0.0000 |
| Positive Predictive Value (TP/(TP + FP)): | 0.4717 |
| Negative Predictive Value (TN/(FN + TN)): | |
| Matthews Correlation Coefficient | |
| ((TP*TN − FP*FN)/sqrt(P*N*P′*N′)): | |
| Area Under Curve (((TP/(TP + FN)) + | 0.5000 |
| (TN/FP + TN))*0.5): | |

TABLE 4
4A Top 72 genes in training data set

| | | |
|---|---|---|
| Real\Predicted | 0 | 1 |
| 0 | 51 | 16 |
| 1 | 7 | 54 |

| | |
|---|---|
| Positive Outcome | 1 |
| Negative Outcome | Outcome(s) other than 1 |
| Actual Positive (P): | 61 |
| Actual Negative (N): | 67 |
| Predictived Positive (P′): | 70 |
| Predictived Negative (N′): | 58 |
| True Positive (TP): | 54 |
| False Positive (FP): | 16 |
| False Negative (FN): | 7 |
| True Negative (TN): | 51 |
| Sensitivity (TP/(TP + FN)): | 0.8852 |
| Specificity (TN/FP + TN)): | 0.7612 |
| Positive Predictive Value (TP/(TP + FP)): | 0.7714 |
| Negative Predictive Value (TN/(FN + TN)): | 0.8793 |
| Matthews Correlation Coefficient | 0.6486 |
| ((TP*TN − FP*FN)/sqrt(P*N*P′*N′)): | |
| Area Under Curve (((TP/(TP + FN)) + | 0.8232 |
| (TN/FP + TN))*0.5): | |

4B Top 72 genes in validation data set

| | | |
|---|---|---|
| Real\Predicted | 0 | 1 |
| 0 | 17 | 11 |
| 1 | 2 | 23 |

| | |
|---|---|
| Positive Outcome | 1 |
| Negative Outcome | Outcome(s) other than 1 |
| Actual Positive (P): | 25 |
| Actual Negative (N): | 28 |
| Predictived Positive (P′): | 34 |
| Predictived Negative (N′): | 19 |
| True Positive (TP): | 23 |
| False Positive (FP): | 11 |
| False Negative (FN): | 2 |
| True Negative (TN): | 17 |
| Sensitivity (TP/(TP + FN)): | 0.9200 |
| Specificity (TN/(FP + TN)): | 0.6071 |
| Positive Predictive Value (TP/(TP + FP)): | 0.6765 |
| Negative Predictive Value (TN/(FN + TN)): | 0.8947 |
| Matthews Correlation Coefficient | 0.5487 |
| ((TP*TN − FP*FN)/sqrt(P*N*P′*N′)): | |
| Area Under Curve (((TP/(TP + FN)) + | 0.7636 |
| (TN/FP + TN))*0.5): | |

TABLE 5
5A Top 77 genes in training data set

| | | |
|---|---|---|
| Real\Predicted | 0 | 1 |
| 0 | 51 | 16 |
| 1 | 8 | 53 |

| | |
|---|---|
| Positive Outcome | 1 |
| Negative Outcome | Outcome(s) other than 1 |
| Actual Positive (P): | 61 |
| Actual Negative (N): | 67 |
| Predictived Positive (P′): | 69 |
| Predictived Negative (N′): | 59 |
| True Positive (TP): | 53 |
| False Positive (FP): | 16 |
| False Negative (FN): | 8 |
| True Negative (TN): | 51 |
| Sensitivity (TP/(TP + FN)): | 0.8689 |
| Specificity (TN/(FP + TN)): | 0.7612 |
| Positive Predictive Value (TP/(TP + FP)): | 0.7681 |
| Negative Predictive Value (TN/(FN + TN)): | 0.8644 |
| Matthews Correlation Coefficient | 0.6313 |
| ((TP*TN − FP*FN)/sqrt(P*N*P′*N′)): | |
| Area Under Curve (((TP/(TP + FN)) + | 0.8150 |
| (TN/FP + TN))*0.5): | |

5B Top 77 genes in validation data set

| | | |
|---|---|---|
| Real\Predicted | 0 | 1 |
| 0 | 17 | 11 |
| 1 | 2 | 23 |

| | |
|---|---|
| Positive Outcome | 1 |
| Negative Outcome | Outcome(s) other than 1 |
| Actual Positive (P): | 25 |
| Actual Negative (N): | 28 |
| Predictived Positive (P′): | 34 |
| Predictived Negative (N′): | 19 |
| True Positive (TP): | 23 |
| False Positive (FP): | 11 |
| False Negative (FN): | 2 |
| True Negative (TN): | 17 |
| Sensitivity (TP/(TP + FN)): | 0.9200 |
| Specificity (TN/(FP + TN)): | 0.6071 |
| Positive Predictive Value (TP/(TP + FP)): | 0.6765 |
| Negative Predictive Value (TN/(FN + TN)): | 0.8947 |
| Matthews Correlation Coefficient | 0.5487 |
| ((TP*TN − FP*FN)/sqrt(P*N*P′*N′)): | |
| Area Under Curve (((TP/(TP + FN)) + | 0.7636 |
| (TN/FP + TN))*0.5): | |

TABLE 6
6A Top 30 genes in training data set

| | |
|---|---|
| Positive Outcome | 1 |
| Negative Outcome | Outcome(s) other than 1 |
| Actual Positive (P): | 25 |
| Actual Negative (N): | 28 |
| Predictived Positive (P′): | 40 |
| Predictived Negative (N′): | 13 |
| True Positive (TP): | 25 |
| False Positive (FP): | 15 |
| False Negative (FN): | 0 |
| True Negative (TN): | 13 |
| Sensitivity (TP/(TP + FN)): | 1.0000 |
| Specificity (TN/(FP + TN)): | 0.4643 |
| Positive Predictive Value (TP/(TP + FP)): | 0.6250 |
| Negative Predictive Value (TN/(FN + TN)): | 1.0000 |
| Matthews Correlation Coefficient | 0.5387 |
| ((TP*TN − FP*FN)/sqrt(P*N*P′*N′)): | |
| Area Under Curve (((TP/(TP + FN)) + | 0.7321 |
| (TN/FP + TN))*0.5): | |

6B Top 58 genes in training data set

| | |
|---|---|
| Positive Outcome | 1 |
| Negative Outcome | Outcome(s) other than 1 |
| Actual Positive (P): | 25 |
| Actual Negative (N): | 28 |
| Predictived Positive (P′): | 36 |
| Predictived Negative (N′): | 17 |
| True Positive (TP): | 23 |
| False Positive (FP): | 13 |
| False Negative (FN): | 2 |
| True Negative (TN): | 15 |
| Sensitivity (TP/(TP + FN)): | 0.9200 |
| Specificity (TN/(FP + TN)): | 0.5357 |
| Positive Predictive Value (TP/(TP + FP)): | 0.6389 |
| Negative Predictive Value (TN/(FN + TN)): | 0.8824 |
| Matthews Correlation Coefficient | 0.4874 |
| ((TP*TN − FP*FN)/sqrt(P*N*P′*N′)): | |
| Area Under Curve (((TP/(TP + FN)) + | 0.7279 |
| (TN/FP + TN))*0.5): | |

6C Top 50 genes in training data set

| | |
|---|---|
| Positive Outcome | 1 |
| Negative Outcome | Outcome(s) other than 1 |
| Actual Positive (P): | 25 |
| Actual Negative (N): | 28 |
| Predictived Positive (P′): | 39 |
| Predictived Negative (N′): | 14 |
| True Positive (TP): | 23 |
| False Positive (FP): | 16 |
| False Negative (FN): | 2 |
| True Negative (TN): | 12 |
| Sensitivity (TP/(TP + FN)): | 0.9200 |
| Specificity (TN/(FP + TN)): | 0.4286 |
| Positive Predictive Value (TP/(TP + FP)): | 0.5897 |
| Negative Predictive Value (TN/(FN + TN)): | 0.8571 |
| Matthews Correlation Coefficient | 0.3947 |
| ((TP*TN − FP*FN)/sqrt(P*N*P′*N′)): | |
| Area Under Curve (((TP/(TP + FN)) + | 0.6743 |
| (TN/FP + TN))*0.5): | |

6D Top 40 genes in training data set

| | |
|---|---|
| Positive Outcome | 1 |
| Negative Outcome | Outcome(s) other than 1 |
| Actual Positive (P): | 25 |
| Actual Negative (N): | 28 |
| Predictived Positive (P′): | 38 |
| Predictived Negative (N′): | 15 |
| True Positive (TP): | 24 |
| False Positive (FP): | 14 |
| False Negative (FN): | 1 |
| True Negative (TN): | 14 |
| Sensitivity (TP/(TP + FN)): | 0.9600 |
| Specificity (TN/(FP + TN)): | 0.5000 |
| Positive Predictive Value (TP/(TP + FP)): | 0.6316 |
| Negative Predictive Value (TN/(FN + TN)): | 0.9333 |
| Matthews Correlation Coefficient | 0.5098 |
| ((TP*TN − FP*FN)/sqrt(P*N*P′*N′)): | |
| Area Under Curve (((TP/(TP + FN)) + | 0.7300 |
| (TN/FP + TN))*0.5): | |

6E 77 genes in training data set for 3 sets of non
overlapping random genes. All yielded the same results.

| | | |
|---|---|---|
| Real\Predicted | 0 | 1 |
| 0 | 30 | 37 |
| 1 | 36 | 25 |

| | |
|---|---|
| Positive Outcome | 1 |
| Negative Outcome | Outcome(s) other than 1 |
| Actual Positive (P): | 61 |
| Actual Negative (N): | 67 |
| Predictived Positive (P′): | 62 |
| Predictived Negative (N′): | 66 |
| True Positive (TP): | 25 |
| False Positive (FP): | 37 |
| False Negative (FN): | 36 |
| True Negative (TN): | 30 |
| Sensitivity (TP/(TP + FN)): | 0.4098 |
| Specificity (TN/(FP + TN)): | 0.4478 |
| Positive Predictive Value (TP/(TP + FP)): | 0.4032 |
| Negative Predictive Value (TN/(FN + TN)): | 0.4545 |
| Matthews Correlation Coefficient | −0.1423 |
| ((TP*TN − FP*FN)/sqrt(P*N*P′*N′)): | |
| Area Under Curve (((TP/(TP + FN)) + | 0.4288 |
| (TN/FP + TN))*0.5): | |

6F 77 genes in validation data set for 3 sets of non
overlapping random genes. All yielded the same results.

| | | |
|---|---|---|
| Real\Predicted | 0 | 1 |
| 0 | 28 | 0 |
| 1 | 0 | 25 |

| | |
|---|---|
| Positive Outcome | 1 |
| Negative Outcome | Outcome(s) other than 1 |
| Actual Positive (P): | 25 |
| Actual Negative (N): | 28 |
| Predictived Positive (P′): | 53 |
| Predictived Negative (N′): | 0 |
| True Positive (TP): | 25 |
| False Positive (FP): | 28 |
| False Negative (FN): | 0 |
| True Negative (TN): | 0 |
| Sensitivity (TP/(TP + FN)): | 1.0000 |
| Specificity (TN/(FP + TN)): | 0.0000 |
| Positive Predictive Value (TP/(TP + FP)): | 0.4717 |
| Negative Predictive Value (TN/(FN + TN)): | |
| Matthews Correlation Coefficient | |
| ((TP*TN − FP*FN)/sqrt(P*N*P′*N′)): | |
| Area Under Curve (((TP/(TP + FN)) + | 0.5000 |
| (TN/FP + TN))*0.5): | |

TABLE 7
Distribution of pCR rates among BRCAness signature
dichotomized groups stratified by HR status

| | V/C (n = 71) | | Control (n = 42) | |
|---|---|---|---|---|
| | Sporadic-like | BRCA1-Like | Sporadic-like | BRCA1-Like |
| | (32) | (39) | (26) | (16) |
| TN (n = 58) | 4/6 | 18/32 | 2/6 | 3/14 |
| HR+HER2− | 1/26 | 4/7 | 4/20 | 0/2 |
| (n = 55) | | | | |

1. A method of assigning treatment to a breast and/or ovarian cancer patient, the method comprising:
determining a level of expression for at least two genes that are selected from Table 1 in a relevant sample from the breast and/or ovarian cancer patient, whereby the sample comprises expression products from a cancer cell of the patient;
comparing said determined level of expression of the at least two genes to the level of expression of the at least two genes in a template;
typing said sample as being BRCA-like or not, based on the comparison of the determined levels of expression; and
assigning DNA-damage inducing treatment to a breast and/or ovarian cancer patient of which the sample is classified as BRCA-like.

2. The method according to claim 1, whereby the sample is typed by determining a level of RNA expression for at least two genes that are selected from Table 1 and comparing said determined RNA level of expression to the level of RNA expression of the at least two genes in a template.

3. The method according to claim 1, whereby the DNA-damage inducing treatment comprises alkylating agents, platinum salts and/or PARP inhibitors.

4. The method according to claim 1, whereby the DNA-damage inducing treatment comprises a nitrogen mustard alkylating agent, N,N′N′-triethylenethiophosphoramide and carboplatin.

5. The method according to claim 1, whereby the DNA-damage inducing treatment comprises a PARP inhibitor.

6. The method according to claim 5, whereby the PARP inhibitor is 2-[(2R)-2-Methylpyrrolidin-2-yl]-1H-benzimidazole-4-carboxamide dihydrochloride benzimidazole carboxamide (ABT-888).

7. The method according to claim 5, whereby the treatment further comprises a tyrosine kinase inhibitor.

8. The method according to claim 7, whereby the tyrosine kinase inhibitor is (2E)-N-[4-[[3-chloro-4-[(pyridin-2-yl)methoxy]phenyl]amino]-3-cyano-7-ethoxyquinolin-6-yl]-4-(dimethylamino)but-2-enamide (Neratinib).

9. The method according to claim 1, whereby a level of expression of at least five genes from Table 1 is determined.

10. The method according to claim 1, wherein the template is a measure of the average level of said at least two genes in at least 10 independent individuals.

11. The method according to claim 1, comprising determining a level of expression for all 77 genes from Table 1 in a relevant sample from the breast and/or ovarian cancer patient.

12. The method according to claim 1, further comprising determining a metastasizing potential of the sample from the patient.

13. The method according to claim 12, whereby the metastasizing potential is determined by a 70 gene Amsterdam profile.
